(19) United States
(12) Patent Application Publication
(10) Pub. No.: US 2024/0041827 A1
(43) Pub. Date: Feb. 8, 2024

(54) COMPOSITIONS FOR THE TREATMENT OF FAT RELATED CONDITIONS AND DISORDERS

(71) Applicant: Raziel Therapeutics Ltd., Rehovot (IL)

(72) Inventors: Alon BLOOMENFELD, Hod Hasharon (IL); Racheli GUETA, Rehovot (IL); Eran BLAUGRUND, Rehovot (IL)

(21) Appl. No.: 18/245,129

(22) PCT Filed: Sep. 15, 2021
(86) PCT No.: PCT/IB2021/000629

§ 371 Date: Mar. 13, 2023

Related U.S. Application Data
(60) Provisional application No. 63/079,243, filed on Sep. 16, 2020.

(51) Int. Cl. A61K 31/403; A61K 38/48; A61P 43/00

(52) U.S. Cl. CPC A61K 31/403; A61K 38/4886; A61P 43/00; C12Y 304/24003

(57) ABSTRACT

The disclosure provides carbazole derivatives in combination with Collagenase Clostridium histolyticum (CCH), and use thereof for the treatment of fat related conditions.

### CROSS REFERENCE

This application claims the benefit of U.S. Provisional Application No. 63/079,243 filed Sep. 16, 2020, which is incorporated by reference herein in its entirety.

### BACKGROUND OF THE INVENTION

Dupuytren's disease, a progressive genetic disorder of pathologic collagen production and deposition, begins with palpable nodules in the palm. Later, pathologic collagen cords form, extend longitudinally, thicken, and shorten, causing flexion contractures of the joints, which can severely limit hand function. Contractures typically affect the metacarpophalangeal joint, the proximal interphalangeal joint, or both. The ring and little fingers are most commonly affected. Dupuytren's disease occurs in all racial and ethnic groups, but the incidence of this disease is highest among people of northern European descent.3-5 The estimated global prevalence among whites is 3 to 6%.

Dupuytren's disease is more common in men than in women, increases in incidence with advancing age, and has been associated with smoking, alcoholism, diabetes, epilepsy, and human immunodeficiency virus infection.

The standard of care for Dupuytren's disease is surgery open fasciectomy (the most common procedure), percutaneous or open fasciotomy, or needle fasciotomy. Surgery is recommended in patients with functional impairment and metacarpophalangeal-joint contractures of 30 degrees or more. For patients with proximal-interphalangeal-joint contractures, recommendations vary and are based on quantitative and qualitative assessments. No effective pharmacotherapy exists.

Collagenase Clostridium histolyticum (CCH), which lyses collagen and leads to disruption of contracted cords, is a new, office-based, minimally invasive, nonsurgical, investigational option for the treatment of advanced Dupuytren's disease. Treatment does not require anesthesia. Collagenase Clostridium histolyticum is injected into the affected cord, and the next day, the treated joint is manipulated to attempt cord rupture. Extensive hand therapy after treatment is not required. In previous single-center studies, injectable collagenase Clostridium histolyticum reduced contractures of the metacarpophalangeal and proximal interphalangeal joints to 0 to 5 degrees of full extension in approximately two thirds of treated joints. A recent phase 3 study also demonstrated the effect of CCH on cellulite, highlighting its effectiveness in reducing subcutaneous fat depositions (enouncement from ENDO at: www.prnewswire.com/news-releases/endo-announces-positive-results-from-phase-3-studies-of-collagenase-clostridium-histolyticum-cch-in-patients-with-cellulite-300745339.html).

### SUMMARY OF THE INVENTION

The present disclosure provides a combination comprising at least two active agents: (1) Collagenase Clostridium histolyticum (CCH); and (2) at least one compound having the general formula (I):

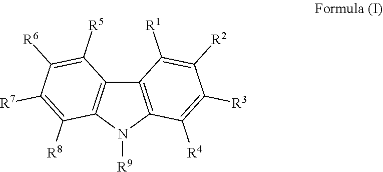

or a pharmaceutically-acceptable salt thereof, wherein:
each of R¹, R², R³, R⁴, R⁵, R⁶, R⁷, and R⁸ is independently H, halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), —ON(═O), C₁₋₅alkyl, C₂₋₅alkenyl, or C₂₋₅alkynyl; wherein each alkyl, alkenyl, or alkynyl is independently optionally substituted with one or more substituents selected from the group consisting of halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), and —ON(═O);
R⁹ is C₁₋₉alkyl, C₂₋₉alkenyl, C₂₋₉alkynyl, or a 3- to 10-membered heterocycloalkyl; wherein R⁹ is substituted with at least one quaternary ammonium group or a phosphonium group;
each R¹⁰ is independently H, C₁₋₅alkyl, C₂₋₅alkenyl, C₂₋₅alkynyl, C₁₋₅heteroalkyl, C₁₋₅haloalkyl, or C₃₋₆cycloalkyl;
each R¹¹ and R¹² is independently H, C₁₋₅alkyl, C₂₋₅alkenyl, C₂₋₅alkynyl, C₁₋₅heteroalkyl, C₁₋₅haloalkyl, or C₃₋₆cycloalkyl; or R¹¹ and R¹² together with the nitrogen atom to which they are attached is a 3- to 10-membered heterocycloalkyl which is optionally substituted; and
each R¹³ is independently H, C₁₋₅alkyl, C₂₋₅alkenyl, C₂₋₅alkynyl, C₁₋₅heteroalkyl, C₁₋₅haloalkyl, or C₃₋₆cycloalkyl.

In some embodiments, R⁹ is C₁-C₉ alkyl substituted with at least one quaternary ammonium group.

In some embodiments, at least one ammonium group is a group of Formula (V):

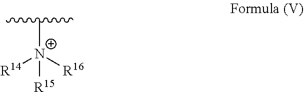

wherein each of R¹⁴, R¹⁵, and R¹⁶ is independently C₁₋₉alkyl, C₂₋₉alkenyl, or C₂₋₉alkynyl.

In some embodiments, the at least one ammonium group is a group of Formula (V′):

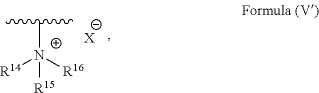

wherein X is a negatively charged ion. In some embodiments, X is a halogen, e.g., Cl.

In some embodiments, each of R¹⁴, R¹⁵, and R¹⁶ is independently methyl. In some embodiments, at least one of R¹, R², R³, and R⁴ is halogen. In some embodiments, at least one of R⁵, R⁶, R⁷, and R⁸ is halogen.

In some embodiments, at least one of R¹, R², R³, and R⁴ is halogen and at least one of R⁵, R⁶, R⁷, and R⁸ is halogen. In some embodiments, the halogen is bromo.

In some embodiments, at least one of R¹, R², R³, and R⁴ is OH. In some embodiments, at least one of R⁵, R⁶, R⁷, and R⁸ is OH. In some embodiments, at least one of R¹, R², R³, and R⁴ is nitro and at least one of R⁵, R⁶, R⁷, and R⁸ is nitro.

In some embodiments a composition according to the invention comprises at least one active agent selected from:

### 3-(3,6-dibromo-9H-carbazol-9-yl)-N,N,N-trimethylpropan-1-aminium

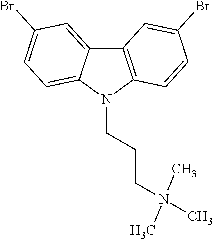 [SMILES: C[N+](C)(C)CCCn1c2ccc(Br)cc2c2cc(Br)ccc21]

### 5-(9H-carbazol-9-yl)-N,N,N-trimethylpentan-1-aminium

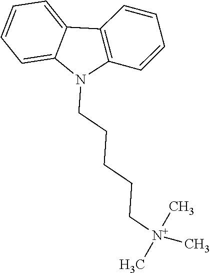 [SMILES: C[N+](C)(C)CCCCCn1c2ccccc2c2ccccc21]

### 5-(2-hydroxy-9H-carbazol-9-yl)-N,N,N-trimethylpentan-1-aminium

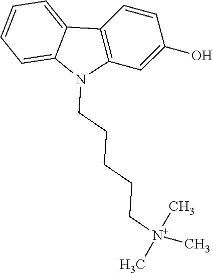 [SMILES: C[N+](C)(C)CCCCCn1c2ccccc2c2ccc(O)cc21]

### 5-(3,6-dibromo-9H-carbazol-9-yl)-N,N,N-trimethylpentan-1-aminium

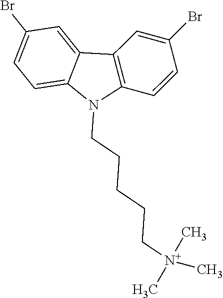 [SMILES: C[N+](C)(C)CCCCCn1c2ccc(Br)cc2c2cc(Br)ccc21]

or any pharmaceutically-acceptable salt thereof.

In some embodiments, the compound of Formula (I) is 5-(3,6-dibromo-9H-carbazol-9-yl)-N,N,N-trimethylpentan-1-aminium chloride.

In some embodiments, the combination comprises at least 0.1% by weight of said at least one compound of formula (I). In some embodiments, the combination comprises from about 0.1% to about 10% by weight of said at least one compound of formula (I). In some embodiments, the combination comprises from about 0.1% to about 5% by weight of said at least one compound of formula (I). In some embodiments, the combination comprises from about 1% to about 5% by weight of said at least one compound of formula (I). In some embodiments, the combination comprises from about 3% to about 5% by weight of said at least one compound of formula (I).

In some embodiments, the combination comprises from about 1 to about 100 mg of said at least one compound of formula (I). In some embodiments, the combination comprises from 50 mg of said at least one compound of formula (I).

In some embodiments, the combination comprises at least 0.1% by weight of said Collagenase Clostridium histolyticum (CCH).

In some embodiments, the CCH and the at least one compound of formula (I) are administered separately.

In some embodiments, the CCH and said at least one compound of formula (I) are administered sequentially, in any order.

In some embodiments, the CCH is administered first. In some embodiments, the compound of formula (I) is administered at least about 1 hour after administration of the CCH. In some embodiments, the compound of formula (I) is administered up to about 24 hours after administration of the CCH. In some embodiments, the compound of formula (I) is administered up to about 48 hours after administration of the CCH.

In some embodiments, the compound of formula (I) is administered first.

In some embodiments, the CCH and said at least one compound of formula (I) are administered concomitantly.

In some embodiments, the CCH and the at least one compound of formula (I) are administered in a single composition. In some embodiments, the CCH and the at least one compound of formula (I) are administered in separate compositions.

In some embodiments, the CCH and at least one compound of general formula (I) are formulated in a single composition.

In some embodiments, the CCH and at least one compound of general formula (I) are formulated in separate compositions.

In some embodiments, the compound of formula (I) is provided in a pharmaceutical composition in unit dosage form, wherein the unit dosage form further comprise sat least one pharmaceutically-acceptable excipient. In some embodiments, the pharmaceutical composition is in a form of a cream, a gel, an ointment, an aqueous solution, an oil-in-water solution, a foam, a patch, a tape, a bandage, a sponge and or combinations thereof. In some embodiments, the pharmaceutical composition is formulated in a liquid dosage form. In some embodiments, the pharmaceutical composition is formulated for parenteral administration. In some embodiments, the pharmaceutical composition is formulated for subcutaneous injection. In some embodiments, the compound of formula (I) is formulated for at least a single injection. In some embodiments, the compound of formula (I) is formulated for multiple injections.

In some embodiments, the CCH is provided in a pharmaceutical composition in unit dosage form, wherein the unit dosage form further comprise sat least one pharmaceutically-acceptable excipient. In some embodiments, the pharmaceutical composition is in a form of a cream, a gel, an ointment, an aqueous solution, an oil-in-water solution, a foam, a patch, a tape, a bandage, a sponge and or combinations thereof. In some embodiments, is formulated for parenteral administration. In some embodiments, the pharmaceutical composition is formulated for subcutaneous injection. In some embodiments, the CCH is formulated for a single injection. In some embodiments, the CCH is formulated for multiple injections.

In some embodiment, the present disclosure provides a combination comprising at least two active agents: (1) Collagenase Clostridium histolyticum (CCH); and (2) at least one compound having the general formula (I) or a pharmaceutically-acceptable salt thereof, for use in the treatment of a fat related condition or disorder of a subject. In some embodiments, the fat related condition or disorder is selected from the group consisting of cellulite, excess fat in various body areas, lipomas, fatty tumor diseases, disorders associated with fat accumulation, Dercum disease (adiposis dolorosa), lipedema, hibernoma and any combinations thereof.

The present disclosure further provides a method of treating a fat related condition or disorder in a patient in need thereof, said method comprising administering to said patient at least one of Collagenase Clostridium histolyticum (CCH) and at least one compound of general formula (I).

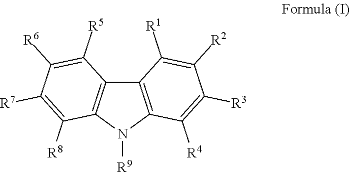

or a pharmaceutically-acceptable salt thereof, wherein:
each of R¹, R², R³, R⁴, R⁵, R⁶, R⁷, and R⁸ is independently H, halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), —ON(═O), C₁₋₅alkyl, C₂₋₅alkenyl, or C₂₋₅alkynyl; wherein each alkyl, alkenyl, or alkynyl is independently optionally substituted with one or more substituents selected from the group consisting of halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), and —ON(═O);
R⁹ is C₁₋₉alkyl, C₂₋₉alkenyl, C₂₋₉alkynyl, or a 3- to 10-membered heterocycloalkyl; wherein R⁹ is substituted with at least one quaternary ammonium group or a phosphonium group;
each R¹⁰ is independently H, C₁₋₅alkyl, C₂₋₅alkenyl, C₂₋₅alkynyl, C₁₋₅heteroalkyl, C₁₋₅haloalkyl, or C₃₋₆cycloalkyl;
each R¹¹ and R¹² is independently H, C₁₋₅alkyl, C₂₋₅alkenyl, C₂₋₅alkynyl, C₁₋₅heteroalkyl, C₁₋₅haloalkyl, or C₃₋₆cycloalkyl; or R¹¹ and R¹² together with the nitrogen atom to which they are attached is a 3- to 10-membered heterocycloalkyl which is optionally substituted; and
each R¹³ is independently H, C₁₋₅alkyl, C₂₋₅alkenyl, C₂₋₅alkynyl, C₁₋₅heteroalkyl, C₁₋₅haloalkyl, or C₃₋₆cycloalkyl.

In some embodiments, the Collagenase Clostridium histolyticum (CCH) is administered to said subject. In some embodiments, the at least one compound of general formula (I) is administered to said subject.

In some embodiments, the compound of formula (I) is administered to fat tissue in the subject at a concentration of least about 100 uM. In some embodiments, the compound of formula (I) is administered to fat tissue in the subject at a concentration of about 100 uM to about 300 uM.

In some embodiments, the compound of Formula (I) is administered at a dose between 40 and 500 mg. In some embodiments, the compound of Formula (I) is administered in multiple injections, wherein each injection comprises between 5 and 7.5 mg of the compound of Formula (I).

In some embodiments, the CCH is administered to fat tissue in the subject at a concentration of greater than about 1 uM. In some embodiments, the CCH is administered to fat tissue in the subject at a concentration of about 1 uM to about 10 uM. In some embodiments, the CCH is administered to fat tissue in the subject at a concentration of about 3 uM.

In some embodiments, each dose of the CCH is about 1 mg or less. In some embodiments, each dose of the CCH is about 0.84 mg.

In some embodiments, the CCH and said at least one compound of formula (I) are administered separately. In some embodiments, the CCH and said at least one compound of formula (I) are administered sequentially, in any order.

In some embodiments, the CCH is administered first. In some embodiments, the compound of formula (I) is administered at least about 1 hour after administration of the CCH. In some embodiments, the compound of formula (I) is administered at least about 24 hours after administration of the CCH. In some embodiments, the compound of formula (I) is administered at least about 48 hours after administration of the CCH. In some embodiments, the compound of formula (I) is administered first.

In some embodiments, the CCH and said at least one compound of formula (I) are administered concomitantly.

In some embodiments, the CCH and said at least one compound of formula (I) are administered in a single composition. In some embodiments, the CCH and said at least one compound of formula (I) are administered in separate compositions.

In some embodiments, the compound of formula (I) is administered parenterally. In some embodiments, the compound of formula (I) is administered subcutaneously.

In some embodiments, the compound of formula (I) is administered in a single injection. In some embodiments, the compound of formula (I) is administered in multiple injections.

In some embodiments, the CCH is administered in a single injection. In some embodiments, the CCH is administered in multiple injections.

In some embodiments, the CCH degrades fibrotic tissue in the subject.

In some embodiments, the compound of formula (I) induces cell death in fat tissue of the subject. In some embodiments, the CCH attenuates compound (I)-induced cell death in non-fat cells adjacent to the fat tissue. In some embodiments, the concentration of CCH sufficient to attenuate compound (I)-induced cell death is about 1 uM or lower. In some embodiments, the concentration of CCH sufficient to attenuate compound (I)-induced cell death is about 0.1 uM to about 1 uM.

In some embodiments, the fat related condition or disorder is selected from the group consisting of cellulite, excess fat in various body areas, lipomas, fatty tumor diseases, disorders associated with fat accumulation, dercum disease, lipedema, hibernoma and any combinations thereof.

In some embodiments, the present disclosure provides a kit comprising at least one combination comprising at least one combination comprising a Collagenase Clostridium histolyticum (CCH) and at least one compound of general formula (I), means for administration of said composition and instructions for use thereof. In some embodiments, the kit further comprises at least one further composition.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The present disclosure provides a combination comprising at least two active agents: (1) Collagenase Clostridium histolyticum (CCH); and (2) at least one compound having the general formula (I), and use thereof for treating a fat related condition or disorder in a subject in need thereof. As contemplated herein, a combination of CCH and a compound of formula (I) is administered to a fatty tissue in a subject. In some embodiments, a compound of formula (I) has a cytotoxic effects on fat cells in the subject. In some embodiments, addition of CCH may enhance therapeutic effect of a compound of formula (I). In some embodiments, addition of CCH may enhance therapeutic effect of a compound of formula (I) by degrading fibrotic tissues in the fat tissue. In some embodiments, CCH is administered to a fatty tissue under conditions sufficient to degrade fibrotic tissue in the fat tissue. A compound of formula (I) is subsequently injected (e.g., 1-48 hours after administration of CCH), thereby inducing fat cell death. In some embodiments, addition of CCH may enhance the effect of a compound of formula (I) at the injection site, as less fibrotic tissue is present.

As demonstrated herein, addition of CCH may attenuate compound (I)-induced cytotoxicity in cells (e.g., non-fat cells) adjacent to the fat tissue. In some embodiments, a combination of compound (I) and CCH is injected into fat tissue, at concentrations sufficient to kill fat cells at the injection site. Both compounds diffuse into adjacent tissues (e.g., non-fat tissues) at lower concentrations, whereby the CCH protects cells in said adjacent tissue from compound (I)-induced cell death. Thus, a combination of CCH and compound (I) on the one hand has improved fat tissue cytotoxicity on one hand, while on the other hand reduces cytotoxicity in non-injected tissue at the vicinity of the injection site, thus minimizing the potential for undesirable side effects at injection-adjacent non-fat tissues, and providing an improved side-effect profile.

### Definitions

Unless defined otherwise, all technical and scientific terms used herein have the same meaning as is commonly understood by one of skill in the art to which this disclosure belongs.

Unless defined otherwise, all technical and scientific terms used herein have the same meaning as is commonly understood by one of skill in the art to which this disclosure belongs.

As used herein, the singular form “a”, “an” and “the” includes plural references unless the context clearly dictates otherwise.

As used herein the term “about” means an amount within 10% of the stated amount, including the stated amount,

The term “C_{x-y}” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons. The term —C_{x-y}alkylene- refers to a substituted or unsubstituted alkylene chain with from x to y carbons in the alkylene chain. For example —C₁₋₆alkylene- may be selected from methylene, ethylene, propylene, butylene, pentylene, and hexylene, any one of which is optionally substituted.

“Alkyl” refers to substituted or unsubstituted saturated hydrocarbon groups, including straight-chain alkyl and branched-chain alkyl groups. An alkyl group may contain from one to twelve carbon atoms (e.g., C₁₋₁₂ alkyl), such as one to eight carbon atoms (C₁₋₈ alkyl) or one to six carbon atoms (C₁₋₆ alkyl). Exemplary alkyl groups include methyl, ethyl, n-propyl, isopropyl, n-butyl, isobutyl, sec-butyl, tert-butyl, pentyl, isopentyl, neopentyl, hexyl, heptyl, octyl, nonyl, and decyl. An alkyl group is attached to the rest of the molecule by a single bond. Unless stated otherwise specifically in the specification, an alkyl group is optionally substituted by one or more substituents such as those substituents described herein. The term “straight or branched C₁-C₅ alkyl” and “straight or branched C₁-C₉ alkyl” encompasses a saturated hydrocarbon chain having between 1 to 5 or 1 to 9 carbon atoms.

“Haloalkyl” refers to an alkyl group that is substituted by one or more halogens. Exemplary haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl.

“Alkenyl” refers to substituted or unsubstituted hydrocarbon groups, including straight-chain or branched-chain alkenyl groups containing at least one double bond. An alkenyl group may contain from two to twelve carbon atoms (e.g., C₂₋₁₂ alkenyl). Exemplary alkenyl groups include ethenyl (i.e., vinyl), prop-1-enyl, but-1-enyl, pent-1-enyl, penta-1,4-dienyl, and the like. Unless stated otherwise specifically in the specification, an alkenyl group is optionally substituted by one or more substituents such as those substituents described herein. The term “straight or branched C₂-C₅ alkenyl” and “straight or branched C₂-C₉ alkenyl” encompasses a hydrocarbon chain having between 1 to 5 or 1 to 9 carbon atoms and at least one double bond

“Alkynyl” refers to substituted or unsubstituted hydrocarbon groups, including straight-chain or branched-chain alkynyl groups containing at least one triple bond. An alkynyl group may contain from two to twelve carbon atoms (e.g., C₂₋₁₂ alkynyl). Exemplary alkynyl groups include ethynyl, propynyl, butynyl, pentynyl, hexynyl, and the like. Unless stated otherwise specifically in the specification, an alkynyl group is optionally substituted by one or more substituents such as those substituents described herein. The term “straight or branched C₂-C₅ alkynyl” and “straight or branched C₂-C₉ alkynyl” encompasses a hydrocarbon chain having between 1 to 5 or 1 to 9 carbon atoms and at least one triple bond “Heteroalkyl”, “heteroalkenyl” and “heteroalkynyl” refer to substituted or unsubstituted alkyl, alkenyl and alkynyl groups which respectively have one or more skeletal chain atoms selected from an atom other than carbon. Exemplary skeletal chain atoms selected from an atom other than carbon include, e.g., O, N, P, Si, S, or combinations thereof, wherein the nitrogen, phosphorus, and sulfur atoms may optionally be oxidized, and the nitrogen heteroatom may optionally be quaternized. If given, a numerical range refers to the chain length in total. For example, a 3- to 8-membered heteroalkyl has a chain length of 3 to 8 atoms. Connection to the rest of the molecule may be through either a heteroatom or a carbon in the heteroalkyl, heteroalkenyl or heteroalkynyl chain. Unless stated otherwise specifically in the specification, a heteroalkyl, heteroalkenyl, or heteroalkynyl group is optionally substituted by one or more substituents such as those substituents described herein.

“Aryl” refers to an aromatic ring wherein each of the atoms forming the ring is a carbon atom. Aryl groups can be optionally substituted. Examples of aryl groups include, but are not limited to, phenyl and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted.

“Heteroaryl” refers to a 3- to 12-membered aromatic ring that comprises at least one heteroatom wherein each heteroatom may be independently selected from N, O, and S. As used herein, the heteroaryl ring may be selected from monocyclic or bicyclic and fused or bridged ring systems wherein at least one of the rings in the ring system is aromatic, i.e., it contains a cyclic, delocalized (4n+2) π-electron system in accordance with the Hückel theory. The heteroatom(s) in the heteroaryl may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, a heteroaryl is optionally substituted by one or more substituents such as those substituents described herein.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted.

The term “heterocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical may be partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

The term “halogen” is meant to encompass any halogen moiety selected from F, Cl, Br and I.

The term “nitro” is a —NO₂ moiety.

The term “amino” refers to —NH₂, —NHR, —NRR′, wherein R, R′ and R″ are each independently selected from straight or branched C₁-C₁₀ alkyl (also termed “alkylamino”), straight or branched C₂-C₁₀ alkenyl, straight or branched C₂-C₁₀ alkynyl. The term amino also includes quarternary ammonium moiety of the form —⁺NRR′R″ wherein R, R′ and R″ are as defined herein above.

The term “nitrilo” refers to —CN,

The term “nitroso” refers to a NO moiety, including C-nitroso moieties (e.g., nitrosoalkanes —R—N═O, wherein R is selected from straight or branched C₁-C₁₀ alkanyl, straight or branched C₂-C₁₀ alkenylene, straight or branched C₂-C₁₀ alkynylene), S-nitroso moieties (nitrosothiols; —S—N═O or —RS—N═O wherein R is selected from straight or branched C₁-C₁₀ alkanyl, straight or branched C₂-C₁₀ alkenylene, straight or branched C₂-C₁₀ alkynylene), N-nitroso moieties (e.g., nitrosamines; —N═N═O, RN—N═O, —RR′N—N═O), and O-nitroso moieties (—O—N═O, —RO—N═O wherein R is selected from straight or branched C₁-C₁₀ alkanyl, straight or branched C₂-C₁₀ alkenylene, straight or branched C₂-C₁₀ alkynylene).

The term “acetyl” refers to a —C(═O)CH₃ moiety.

The terms “acetamido” and “acylamido” refers to —CH₂C(═O)NH₂ and CH₃C(═O)NH-respectively.

The term “straight or branched C₁-C₅ alkoxy” is meant to encompass an —OR moiety wherein R is selected from a straight or branched C₁-C₅ alkyl, straight or branched C₂-C₅ alkenyl and straight or branched C₂-C₅ alkynyl.

The term “straight or branched C₁-C₅ carboxyl” refers to a —R—C(═O)OH moiety wherein R is selected from a straight or branched C₁-C₅ alkanyl, straight or branched C₂-C₅ alkenylene and straight or branched C₂-C₅ alkynylene.

The term “straight or branched C₁-C₅ ester” refers to a RC(═O)O— moiety wherein R is selected from a straight or branched C₁-C₅ alkyl, straight or branched C₂-C₅ alkenyl and straight or branched C₂-C₅ alkynyl.

The term “straight or branched C₁-C₅ thioxy” refers to a RS— moiety wherein R is selected from a straight or branched C₁-C₅ alkyl, straight or branched C₂-C₅ alkenyl and straight or branched C₂-C₅ alkynyl.

The term “straight or branched C₁-C₅ sulfinyl” and “straight or branched C₁-C₅ thionyl” refers to a RS(═O)— moiety wherein R is selected from a straight or branched C₁-C₅ alkyl, straight or branched C₂-C₅ alkenyl and straight or branched C₂-C₅ alkynyl.

The term “phosphonium” refers to a —P+RR′R″ moiety wherein R, R′ and R″ are each selected from a straight or branched C₁-C₁₀ alkyl, straight or branched C₂-C₁₀ alkenyl and straight or branched C₂-C₁₀ alkynyl.

The term “piperazinyl” encompasses a moiety selected from:

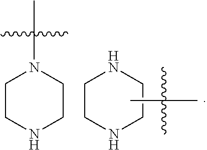

The term “pyridinyl” encompasses a moiety:

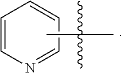

The term “piperidinyl” encompasses a moiety selected from:

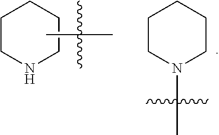

The term “morpholinyl” encompasses a moiety selected from:

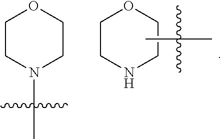

The term “thiomorpholinyl” encompasses a moiety selected from:

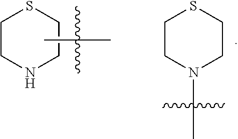

As used herein, [symbol] denotes a point of attachment of the respective moiety to the rest of the molecule.

The term “substituted” refers to moieties having substituents replacing a hydrogen on one or more carbons or heteroatoms of the structure. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, a carbocycle, a heterocycle, a cycloalkyl, a heterocycloalkyl, an aromatic and heteroaromatic moiety.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants.

Where substituent groups are specified by their conventional chemical formulae, written from left to right, they equally encompass the chemically identical substituents that would result from writing the structure from right to left, e.g., —CH₂O— is equivalent to —OCH₂—.

“Optional” or “optionally” means that the subsequently described event of circumstances may or may not occur, and that the description includes instances where the event or circumstance occurs and instances in which it does not. For example, “optionally substituted aryl” means that the aryl group may or may not be substituted and that the description includes both substituted aryl groups and aryl groups having no substitution.

Compounds of the present disclosure also include crystalline and amorphous forms of those compounds, pharmaceutically-acceptable salts, zwitterions, prodrugs and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. For example, hydrogen has three naturally occurring isotopes, denoted ¹H (protium), ²H (deuterium), and ³H (tritium). Protium is the most abundant isotope of hydrogen in nature. Enriching for deuterium may afford certain therapeutic advantages, such as increased in vivo half-life and/or exposure, or may provide a compound useful for investigating in vivo routes of drug elimination and metabolism. Isotopically-enriched compounds may be prepared by conventional techniques well known to those skilled in the art.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)— or (S)—. The present chemical entities, pharmaceutical compositions and methods are meant to include all such possible stereoisomers, including racemic mixtures, optically pure forms, mixtures of diastereomers and intermediate mixtures. Optically active (R)- and (S)-isomers can be prepared using chiral synthons or chiral reagents or resolved using conventional techniques. The optical activity of a compound can be analyzed via any suitable method, including but not limited to chiral chromatography and polarimetry, and the degree of predominance of one stereoisomer over the other isomer can be determined.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, chemical entities described herein are intended to include all Z-, E- and tautomeric forms as well.

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

When stereochemistry is not specified, certain small molecules described herein include, but are not limited to, when possible, their isomers, such as enantiomers and diastereomers, mixtures of enantiomers, including racemates, mixtures of diastereomers, and other mixtures thereof, to the extent they can be made by one of ordinary skill in the art by routine experimentation. In those situations, the single enantiomers or diastereomers, i.e., optically active forms, can be obtained by asymmetric synthesis or by resolution of the racemates or mixtures of diastereomers. Resolution of the racemates or mixtures of diastereomers, if possible, can be accomplished, for example, by conventional methods such as crystallization in the presence of a resolving agent, or chromatography, using, for example, a chiral high-pressure liquid chromatography (HPLC) column. Furthermore, a mixture of two enantiomers enriched in one of the two can be purified to provide further optically enriched form of the major enantiomer by recrystallization and/or trituration. In addition, such certain small molecules include Z- and E-forms (or cis- and trans-forms) of certain small molecules with carbon-carbon double bonds or carbon-nitrogen double bonds. Where certain small molecules described herein exist in various tautomeric forms, the term “certain small molecule” is intended to include all tautomeric forms of the certain small molecule.

As used herein, “treatment” or “treating” refers to an approach for obtaining beneficial or desired results with respect to a disease, disorder, or medical condition including but not limited to a therapeutic benefit and/or a prophylactic benefit. A therapeutic benefit can include, for example, the eradication or amelioration of the underlying disorder being treated. Also, a therapeutic benefit can include, for example, the eradication or amelioration of one or more of the physiological symptoms associated with the underlying disorder such that an improvement is observed in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made.

The term “effective amount” or “therapeutically effective amount” refers to that amount of a compound described herein that is sufficient to affect the intended application, including but not limited to disease treatment, including alleviation of symptoms, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “co-administration,” “administered in combination with,” and their grammatical equivalents, as used herein, encompass administration of two or more agents to an animal, including humans, so that both agents and/or their metabolites are present in the subject at the same time. Co-administration includes simultaneous administration in separate compositions, administration at different times in separate compositions, or administration in a composition in which both agents are present.

### Combinations

The present disclosure provides a combination comprising at least two active agents: (1) Collagenase Clostridium histolyticum (CCH); and (2) at least one compound having the general formula (I), as described herein.

In some embodiments, a combination of the disclosure comprises a single composition comprising both active agents (1) and (2) as defined herein.

In other embodiments, a combination of the invention comprises at least two separate compositions, each comprising a separate active agent. Thus, a combination of the invention comprises, in some embodiments, at least one first composition comprising active agent (1) Collagenase Clostridium histolyticum (CCH) and at least one second composition comprising active agent (2), a compound of general formula (I).

In some embodiments, a composition of the invention comprises at least 0.1% by weight of said at least one compound of formula (I) of total composition. In some embodiments, the combination comprises from about 0.1% to about 10% by weight of said at least one compound of formula (I). In some embodiments, the combination comprises from about 0.1% to about 5% by weight of said at least one compound of formula (I).

In some embodiments, the combination comprises from about 1 to about 100 mg of said at least one compound of formula (I). In some embodiments, the combination comprises from 50 mg of said at least one compound of formula (I).

In other embodiments, a composition of the invention comprises at least 1% by weight of said Collagenase Clostridium histolyticum (CCH) of the total composition.

In some embodiments, the CCH and the at least one compound of formula (I) are administered separately.

In some embodiments, the CCH and said at least one compound of formula (I) are administered sequentially, in any order.

In some embodiments, the CCH is administered first. In some embodiments, the compound of formula (I) is administered at least about 1 hour after administration of the CCH. In some embodiments, the compound of formula (I) is administered up to about 48 hours after administration of the CCH.

In some embodiments, the compound of formula (I) is administered first.

In some embodiments, the CCH and said at least one compound of formula (I) are administered concomitantly.

In some embodiments, the CCH and the at least one compound of formula (I) are administered in a single composition. In some embodiments, the CCH and the at least one compound of formula (I) are administered in separate compositions.

In some embodiments, the CCH and at least one compound of general formula (I) are formulated in a single composition. In some embodiments, the CCH and at least one compound of general formula (I) are formulated in separate compositions.

The compound of Formula (I) is represented by the structure:

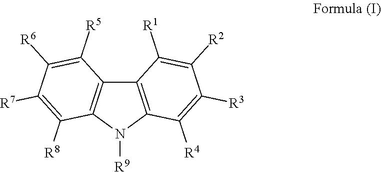

or a pharmaceutically-acceptable salt thereof, wherein:
each of R¹, R², R³, R⁴, R⁵, R⁶, R⁷, and R⁸ is independently H, halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), —ON(═O), C₁₋₅alkyl, C₂₋₅alkenyl, or C₂₋₅alkynyl; wherein each alkyl, alkenyl, or alkynyl is independently optionally substituted with one or more substituents selected from the group consisting of halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), and —ON(═O);
R⁹ is C₁₋₉alkyl, C₂₋₉alkenyl, C₂₋₉alkynyl, or a 3- to 10-membered heterocycloalkyl; wherein R⁹ is substituted with at least one quaternary ammonium group or a phosphonium group;
each R¹⁰ is independently H, C₁₋₅alkyl, C₂₋₅alkenyl, C₂₋₅alkynyl, C₁₋₅heteroalkyl, C₁₋₅haloalkyl, or C₃₋₆cycloalkyl;
each R¹¹ and R¹² is independently H, C₁₋₅alkyl, C₂₋₅alkenyl, C₂₋₅alkynyl, C₁₋₅heteroalkyl, C₁₋₅haloalkyl, or C₃₋₆cycloalkyl; or R¹¹ and R¹² together with the nitrogen atom to which they are attached is a 3- to 10-membered heterocycloalkyl which is optionally substituted; and
each R¹³ is independently H, C₁₋₅alkyl, C₂₋₅alkenyl, C₂₋₅alkynyl, C₁₋₅heteroalkyl, C₁₋₅haloalkyl, or C₃₋₆cycloalkyl.

In some embodiments, each of R¹, R², R³, R⁴, R⁵, R⁶, R⁷, and R⁸ is independently selected from the group consisting of H, halogen, —CN, —NO₂, —OR¹⁰, —S(═O)₂R¹⁰, —NR¹¹R¹², —C(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², C₁₋₅alkyl, C₂₋₅alkenyl, and C₂₋₅alkynyl; wherein each alkyl, alkenyl, and alkynyl is independently optionally substituted with one or more substituents selected from halogen, —CN, —NO₂, —OR¹⁰, —S(═O)₂R¹⁰, —NR¹¹R¹², —C(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, and —NR¹³S(═O)₂NR¹¹R¹².

In some embodiments, each of R¹, R², R³, R⁴, R⁵, R⁶, R⁷, and R⁸ is independently selected from the group consisting of H, halogen, —CN, —NO₂, —OR¹⁰, —NR¹¹R¹², —C(═O)R¹⁰, —C(═O)OR¹⁰, and C₁₋₅alkyl; wherein each alkyl is independently optionally substituted with one or more substituents selected from halogen, —CN, —NO₂, —OR¹⁰, —S(═O)₂R¹⁰, —NR¹¹R¹², —C(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, and —NR¹³S(═O)₂NR¹¹R¹².

In some embodiments, each of R¹, R², R³, R⁴, R⁵, R⁶, R⁷, and R⁸ is independently selected from the group consisting of H, halogen, —CN, —NO₂, —OR¹⁰, and —NR¹¹R¹².

In some embodiments, each of R¹, R², R³, R⁴, R⁵, R⁶, R⁷, and R⁸ is hydrogen.

In some embodiments, at least one of R¹, R², R³, and R⁴ is halogen. In some embodiments, R¹ is halogen. In some embodiments, R² is halogen. In some embodiments, R³ is halogen. In some embodiments, R⁴ is halogen. In some embodiments, at least one of R⁵, R⁶, R⁷, and R⁸ is halogen. In some embodiments, R⁵ is halogen. In some embodiments, R⁶ is halogen. In some embodiments, R⁷ is halogen. In some embodiments, R⁸ is halogen. In some embodiments, at least one of R¹, R², R³, and R⁴ is halogen and at least one of R⁵, R⁶, R⁷, and R⁸ is halogen. In some embodiments, at least one of R¹, R², R³, and R⁴ is halogen, at least one of R⁵, R⁶, R⁷, and R⁸ is halogen, and the rest of R¹, R², R³, R⁴, R⁵, R⁶, R⁷, and R⁸ are hydrogen. In some embodiments, R¹ is halogen and R⁵ is halogen. In some embodiments, R¹ is halogen and R⁶ is halogen. In some embodiments, R¹ is halogen and R⁷ is halogen. In some embodiments, R¹ is halogen and R⁸ is halogen. In some embodiments, R² is halogen and R⁵ is halogen. In some embodiments, R² is halogen and R⁶ is halogen. In some embodiments, R² is halogen and R⁷ is halogen. In some embodiments, R² is halogen and R⁸ is halogen. In some embodiments, R³ is halogen and R⁵ is halogen. In some embodiments, R³ is halogen and R⁶ is halogen. In some embodiments, R³ is halogen and R⁷ is halogen. In some embodiments, R³ is halogen and R⁸ is halogen. In some embodiments, R⁴ is halogen and R⁵ is halogen. In some embodiments, R⁴ is halogen and R⁶ is halogen. In some embodiments, R⁴ is halogen and R⁷ is halogen. In some embodiments, R⁴ is halogen and R⁸ is halogen.

In some embodiments, the halogen is bromo. In some embodiments, the halogen is chloro. In some embodiments, the halogen is fluoro. In some embodiments, the halogen is iodo.

In some embodiments, at least one of R¹, R², R³, and R⁴ is OH. In some embodiments, R¹ is OH. In some embodiments, R² is OH. In some embodiments, R³ is OH. In some embodiments, R⁴ is OH. In some embodiments, at least one of R⁵, R⁶, R⁷, and R⁸ is OH. In some embodiments, R⁵ is OH. In some embodiments, R⁶ is OH. In some embodiments, R⁷ is OH. In some embodiments, R⁸ is OH. In some embodiments, at least one of R¹, R², R³, R⁴, R⁵, R⁶, R⁷, and R⁸ is OH, and the rest of R¹, R², R³, R⁴, R⁵, R⁶, R⁷, and R⁸ are hydrogen.

In some embodiments, at least one of R¹, R², R³, and R⁴ is nitro and at least one of R⁵, R⁶, R⁷, and R⁸ is nitro. In some embodiments, at least one of R¹, R², R³, R⁴, R⁵, R⁶, R⁷, and R⁸ is nitro, and the rest of R¹, R², R³, R⁴, R⁵, R⁶, R⁷, and R⁸ are hydrogen. In some embodiments, R¹ is nitro and R⁵ is nitro. In some embodiments, R¹ is nitro and R⁶ is nitro. In some embodiments, R¹ is nitro and R⁷ is nitro. In some embodiments, R¹ is nitro and R⁸ is nitro. In some embodiments, R² is halogen and R⁵ is nitro. In some embodiments, R² is nitro and R⁶ is nitro. In some embodiments, R² is halogen and R⁷ is nitro. In some embodiments, R² is nitro and R⁸ is nitro. In some embodiments, R³ is halogen and R⁵ is nitro. In some embodiments, R³ is nitro and R⁶ is nitro. In some embodiments, R³ is halogen and R⁷ is nitro. In some embodiments, R³ is nitro and R⁸ is nitro. In some embodiments, R⁴ is halogen and R⁵ is nitro. In some embodiments, R⁴ is nitro and R⁶ is nitro. In some embodiments, R⁴ is halogen and R⁷ is nitro. In some embodiments, R⁴ is nitro and R⁸ is nitro.

In some embodiments, R⁹ is selected from the group consisting of C₁₋₉alkyl, C₂₋₉alkenyl, C₂₋₉ alkynyl, and 3- to 10-membered heterocycloalkyl. In some embodiments, R⁹ is C₁₋₉alkyl which is optionally substituted. In some embodiments, R⁹ is a C₁₋₉alkyl which is substituted with at least one quaternary ammonium group. In some embodiments, R⁹ is a C₁₋₉alkyl which is substituted with at least one phosphonium group. In some embodiments, R⁹ is C₂₋₉alkenyl substituted with at least one phosphonium group. In some embodiments, R⁹ is C₂₋₉alkenyl substituted with at least one quaternary ammonium group. In some embodiments, R⁹ is C₂₋₉alkynyl substituted with at least one phosphonium group. In some embodiments, R⁹ is C₂₋₉alkynyl substituted with at least one quaternary ammonium group. In some embodiments, R⁹ is a 3- to 10-membered heterocycloalkyl. In some embodiments, R⁹ is piperazinyl. In some embodiments, R⁹ is pyridinyl. In some embodiments, R⁹ is piperidinyl. In some embodiments, R⁹ is morpholinyl. In some embodiments, R⁹ is thiomorpholinyl. In some embodiments, R⁹ is C₁₋₉alkyl substituted with at least one phosphonium group. In some embodiments, R⁹ is C₁₋₉alkyl substituted with at least one quaternary ammonium group. In some embodiments, R⁹ is propyl substituted with at least one quaternary ammonium group. In some embodiments, R⁹ is butyl substituted with at least one quaternary ammonium group. In some embodiments, R⁹ is pentyl substituted with at least one quaternary ammonium group.

In some embodiments, a compound comprises a positively charged moiety (e.g., ammonium, phosphonium). In some embodiments, a compound which comprises a positively charged moiety may be in the form of a salt, further comprising a negatively-charged counter-ion. For example, when a compound comprises a quaternary ammonium or a phosphonium salt, the compound may be in the form of a salt with a negatively-charged counter-ion, such as a halide (e.g., chloride).

In some embodiments, a compound comprises a negatively charged moiety. In some embodiments, a compound which comprises a negatively charged moiety may be in the form of a salt, further comprising a positively-charged counter-ion.

In some embodiments, the at least one quaternary ammonium group is represented by the structure of Formula (V):

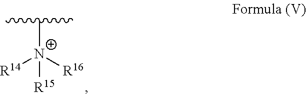

wherein each of R¹⁴, R¹⁵, and R¹⁶ is independently selected from the group consisting of C₁₋₉alkyl, C₂₋₉alkenyl, and C₂₋₉alkynyl. In some embodiments, each of R¹⁴, R¹⁵, and R¹⁶ is independently C₂₋₉alkenyl. In some embodiments, each of R¹⁴, R¹⁵, and R¹⁶ is independently C₂₋₉ alkynyl. In some embodiments, each of R¹⁴, R¹⁵, and R¹⁶ is independently C₁₋₉alkyl. In some embodiments, each of R¹⁴, R¹⁵, and R¹⁶ is methyl. In some embodiments, Formula (V) further comprises a counter-ion, as is represented by the structure of formula (V′):

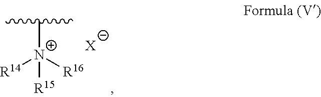

wherein X^{⊖} is a negatively charged counter-ion as defined herein. In some embodiments, X is halogen, e.g., Cl, Br, F, I. In one embodiment, X is a halide, e.g., Cl.

In some embodiments, the at least one phosphonium group is of Formula (VI):

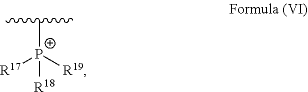

wherein each of R¹⁷, R¹⁸, and R¹⁹ is independently selected from the group consisting of C₁₋₉alkyl, C₂₋₉alkenyl, and C₂₋₉alkynyl. In some embodiments, each of R¹⁷, R¹⁸, and R¹⁹ is independently C₂₋₉alkenyl. In some embodiments, each of R¹⁷, R¹⁸, and R¹⁹ is independently C₂₋₉alkynyl. In some embodiments, each of R¹⁷, R¹⁸, and R¹⁹ is independently C₁₋₉alkyl. In some embodiments, each of R¹⁷, R¹⁸, and R¹⁹ is methyl. In some embodiments, Formula (VI) further comprises a counter-ion, as is represented by the structure of formula (VI′):

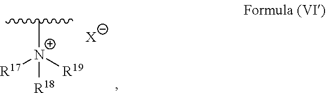

wherein X^{⊖} is a negatively charged counter-ion as defined herein. In some embodiments, X is halogen, e.g., Cl, Br, F, I. In one embodiment, X is a halide, e.g., Cl.

In some embodiments, R¹⁰ is independently selected from H, C₁₋₅alkyl, C₂₋₅alkenyl, C₂₋₅alkynyl, C₁₋₅heteroalkyl, C₁₋₅haloalkyl, and C₃₋₆cycloalkyl. In some embodiments, R¹⁰ is independently selected from H, C₁₋₅alkyl, C₁₋₅heteroalkyl, C₁₋₅haloalkyl, and C₃₋₆cycloalkyl. In some embodiments, R¹⁰ is independently selected from H, C₁₋₅alkyl, and C₃₋₆cycloalkyl. In some embodiments, R¹⁰ is H. In some embodiments, R¹⁰ is independently C₁₋₅alkyl. In some embodiments, R¹⁰ is independently C₂₋₅alkenyl. In some embodiments, R¹⁰ is independently C₂₋₅alkynyl. In some embodiments, R¹⁰ is independently C₁₋₅heteroalkyl. In some embodiments, R¹⁰ is independently C₁₋₅haloalkyl. In some embodiments, R¹⁰ is independently C₃₋₆cycloalkyl.

In some embodiments, R¹¹ and R¹² is independently selected from H, C₁₋₅alkyl, C₂₋₅alkenyl, C₂₋₅alkynyl, C₁₋₅heteroalkyl, C₁₋₅haloalkyl, and C₃₋₆cycloalkyl. In some embodiments, R¹¹ and R¹² together along with the nitrogen atom to which they are attached may form a 3- to 10-membered heterocycloalkyl, which may optionally be substituted. In some embodiments, R¹¹ and R¹² are each independently selected from the group consisting of H, C₁₋₅alkyl, C₂₋₅alkenyl, C₂₋₅alkynyl, C₁₋₅heteroalkyl, C₁₋₅haloalkyl, and C₃₋₆cycloalkyl. In some embodiments, each of R¹¹ and R¹² is independently selected from H, C₁₋₅alkyl, C₁₋₅heteroalkyl, C₁₋₅haloalkyl, and C₃₋₆cycloalkyl. In some embodiments, each of R¹¹ and R¹² is independently selected from H, C₁₋₅alkyl, and C₃₋₆ cycloalkyl. In some embodiments, R¹¹ and R¹² are both H. In some embodiments, each R¹¹ and R¹² is independently C₁₋₅alkyl. In some embodiments, each R¹¹ and R¹² is independently C₂₋₅alkenyl. In some embodiments, each R¹¹ and R¹² is independently C₂₋₅alkynyl. In some embodiments, each R¹¹ and R¹² is independently C₁₋₅heteroalkyl. In some embodiments, each R¹¹ and R¹² is independently C₁₋₅haloalkyl. In some embodiments, each R¹¹ and R¹² is independently C₃₋₆cycloalkyl. In some embodiments, R¹¹ and R¹² are taken together along with the nitrogen atom to which they are attached to form a 3- to 10-membered heterocycloalkyl, which may optionally be substituted.

In some embodiments, R¹³ is independently selected from H, C₁₋₅alkyl, C₂₋₅alkenyl, C₂₋₅alkynyl, C₁₋₅heteroalkyl, C₁₋₅haloalkyl, and C₃₋₆cycloalkyl. In some embodiments, R¹³ is independently selected from H, C₁₋₅alkyl, C₁₋₅heteroalkyl, C₁₋₅haloalkyl, and C₃₋₆cycloalkyl. In some embodiments, R¹³ is independently selected from H, C₁₋₅alkyl, and C₃₋₆cycloalkyl. In some embodiments, R¹³ is H. In some embodiments, R¹³ is independently C₁₋₅alkyl. In some embodiments, R¹³ is independently C₂₋₅alkenyl. In some embodiments, R¹³ is independently C₂₋₅alkynyl. In some embodiments, R¹³ is independently C₁₋₅heteroalkyl. In some embodiments, R¹³ is independently C₁₋₅haloalkyl. In some embodiments, R¹³ is independently C₃₋₆cycloalkyl.

In some embodiments, the compound of Formula (I) is selected from the group consisting of.
3-(3,6-dibromo-9H-carbazol-9-yl)-N,N,N-trimethylpropan-1-aminium,
5-(9H-carbazol-9-yl)-N,N,N-trimethylpentan-1-aminium,
5-(2-hydroxy-9H-carbazol-9-yl)-N,N,N-trimethylpentan-1-aminium, and
5-(3,6-dibromo-9H-carbazol-9-yl)-N,N,N-trimethylpentan-1-aminium.

In some embodiments, the compound of Formula (I) is 3-(3,6-dibromo-9H-carbazol-9-yl)-N,N,N-trimethylpropan-1-aminium. In some embodiments, the compound of Formula (I) is 5-(9H-carbazol-9-yl)-N,N,N-trimethylpentan-1-aminium. In some embodiments, the compound of Formula (I) is 5-(2-hydroxy-9H-carbazol-9-yl)-N,N,N-trimethylpentan-1-aminium. In some embodiments, the compound of Formula (I) is 5-(3,6-dibromo-9H-carbazol-9-yl)-N,N,N-trimethylpentan-1-aminium.

In some embodiments, the compound of formula (I) is in a pharmaceutically-acceptable salt form. The term “salt” or “pharmaceutically-acceptable salt” refers to salts derived from a variety of organic and inorganic counter-ions. When a compound of formula (I) contains one or more positive charges, the counter-ion has the corresponding one or more negative charges, generating a neutral molecule. When a compound of formula (I) contains one or more negative charges, the counter-ion has the corresponding one or more positive charges, generating a neutral molecule.

Pharmaceutically-acceptable acid addition salts can be formed with inorganic acids and organic acids and their corresponding counter-ions. Inorganic acids from which salts can be derived include, for example, hydrochloric acid, hydrobromic acid, sulfuric acid, nitric acid, phosphoric acid, and the like. For example, salts may include a counter anion being a halogen counter-anion such as for example chloride and bromide anions. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically-acceptable base addition salts can be formed with inorganic and organic bases and their corresponding counter-ions. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically-acceptable base addition salt is chosen from ammonium, phosphonium, potassium, sodium, calcium, and magnesium salts.

In some embodiments, Compound (I) is in the form of a salt with an inorganic acid. In some embodiments, Compound (I) is in the form of a salt with a hydrochloric acid (i.e., the counter-ion is chloride (Cl⁻)).

In some embodiments, the compound of Formula (I) is 5-(3,6-dibromo-9H-carbazol-9-yl)-N,N,N-trimethylpentan-1-aminium, represented by the structure of formula 1:

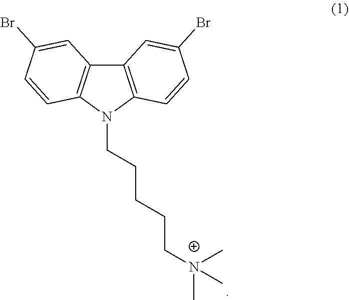 [SMILES: C[N+](C)(C)CCCCCn1c2ccc(Br)cc2c2cc(Br)ccc21]

In some embodiments, the compound of Formula (I) is 5-(3,6-dibromo-9H-carbazol-9-yl)-N,N,N-trimethylpentan-1-aminium chloride, represented by the structure of formula 1A:

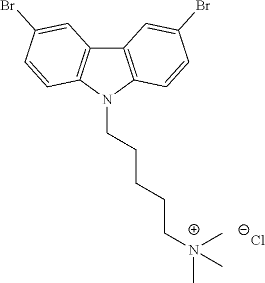 [SMILES: C[N+](C)(C)CCCCCn1c2ccc(Br)cc2c2cc(Br)ccc21 ; [Cl-]]

In some embodiments, the compound of Formula (I) is represented by the structure of formula (2):

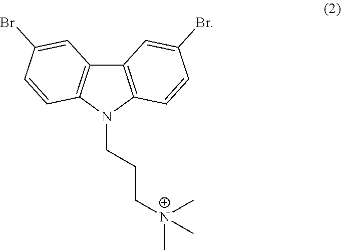 [SMILES: C[N+](C)(C)CCCn1c2ccc(Br)cc2c2cc(Br)ccc21]

In some embodiments, the compound of Formula (I) is represented by the structure of formula (3):

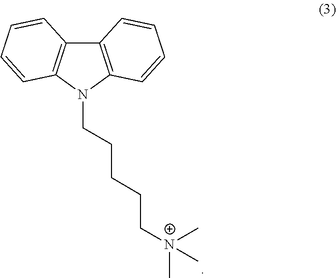 [SMILES: C[N+](C)(C)CCCCCn1c2ccccc2c2ccccc21]

In some embodiments, the compound of Formula (I) is represented by the structure of formula (4):

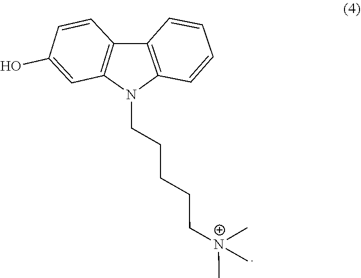 [SMILES: C[N+](C)(C)CCCCCn1c2ccccc2c2ccc(O)cc21]

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

In some embodiments, the compound of formula (I) is represented by formula (I-A). Thus, in some embodiments, the present disclosure provides a combination comprising at least two active agents: (1) Collagenase Clostridium histolyticum (CCH); and (2) at least one compound having the general formula (I-A) or any salt thereof. The compound of formula (I-A) is represented by the structure:

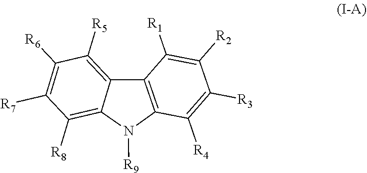

wherein each of R₁-R₈ is independently selected from the group consisting of H, OH, SH, halogen, nitro, amino, nitrilo, nitroso, acetyl, acetamido, acylamido, alkylamino, straight or branched C₁-C₅ alkyl, straight or branched C₂-C₅alkenyl, straight or branched C₂-C₅alkynyl, amine, straight or branched C₁-C₅alkoxy, straight or branched C₁-C₅ carboxyl, straight or branched C₁-C₅ ester, straight or branched C₁-C₅thioxy, straight or branched C₁-C₅sulfinyl, straight or branched C₁-C₅thionyl; R₉ is selected from straight or branched C₁-C₉ alkyl, straight or branched C₂-C₉alkenyl, straight or branched C₂-C₉alkynyl, piperazinyl, pyridinyl, piperidinyl, morpholinyl and thiomorpholinyl; R₉ is substituted with at least one quaternary amino (ammonium) group or a phophonium group.

### Methods of Use

The present disclosure further provides a method of treating a fat related condition or disorder in a patient in need thereof, said method comprising administering to said patient at least one of Collagenase Clostridium histolyticum (CCH) and at least one compound of general formula (I):

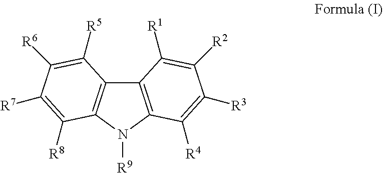

or a pharmaceutically-acceptable salt thereof, wherein:
each of R¹, R², R³, R⁴, R⁵, R⁶, R⁷, and R⁸ is independently H, halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), —ON(═O), C₁₋₅alkyl, C₂₋₅alkenyl, or C₂₋₅alkynyl; wherein each alkyl, alkenyl, or alkynyl is independently optionally substituted with one or more substituents selected from the group consisting of halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), and —ON(═O);
R⁹ is C₁₋₉alkyl, C₂₋₉alkenyl, C₂₋₉alkynyl, or a 3- to 10-membered heterocycloalkyl; wherein R⁹ is substituted with at least one quaternary ammonium group or a phosphonium group;
each R¹⁰ is independently H, C₁₋₅alkyl, C₂₋₅alkenyl, C₂₋₅alkynyl, C₁₋₅heteroalkyl, C₁₋₅haloalkyl, or C₃₋₆cycloalkyl;
each R¹¹ and R¹² is independently H, C₁₋₅alkyl, C₂₋₅alkenyl, C₂₋₅alkynyl, C₁₋₅heteroalkyl, C₁₋₅haloalkyl, or C₃₋₆cycloalkyl; or R¹¹ and R¹² together with the nitrogen atom to which they are attached is a 3- to 10-membered heterocycloalkyl which is optionally substituted; and
each R¹³ is independently H, C₁₋₅alkyl, C₂₋₅alkenyl, C₂₋₅alkynyl, C₁₋₅heteroalkyl, C₁₋₅haloalkyl, or C₃₋₆cycloalkyl.

The present disclosure further provides a method of treating a fat related condition or disorder in a patient in need thereof, said method comprising administering to said patient at least one of Collagenase Clostridium histolyticum (CCH) and at least one compound of general formula (I-A):

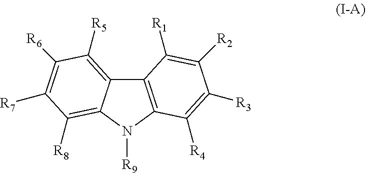

wherein each of R₁-R₈ is independently selected from the group consisting of H, OH, SH, halogen, nitro, amino, nitrilo, nitroso, acetyl, acetamido, acylamido, alkylamino, straight or branched C₁-C₅ alkyl, straight or branched C₂-C₅alkenyl, straight or branched C₂-C₅alkynyl, amine, straight or branched C₁-C₅alkoxy, straight or branched C₁-C₅ carboxyl, straight or branched C₁-C₅ ester, straight or branched C₁-C₅thioxy, straight or branched C₁-C₅sulfinyl, straight or branched C₁-C₅thionyl; R₉ is selected from straight or branched C₁-C₉ alkyl, straight or branched C₂-C₉alkenyl, straight or branched C₂-C₉alkynyl, piperazinyl, pyridinyl, piperidinyl, morpholinyl and thiomorpholinyl; R₉ is substituted with at least one quaternary amino (ammonium) group or a phophonium group.

The present disclosure further provides a composition comprising at least one of Collagenase Clostridium histolyticum (CCH) and at least one compound having the general formula (I) or any salt thereof, for use in the treatment of a fat related condition or disorder (including an aesthetic condition or disorder and a diseased condition or disorder).

The present disclosure further provides a composition comprising at least one of Collagenase Clostridium histolyticum (CCH) and at least one compound having the general formula (I-A) or any salt thereof, for use in the treatment of a fat related condition or disorder (including an aesthetic condition or disorder and a diseased condition or disorder).

In some embodiments, the fat related condition or disorder is selected from the group consisting of cellulite, excess fat in various body areas, lipomas, fatty tumor diseases, disorders associated with fat accumulation, Dercum disease (Adiposis Dolorosa), lipedema, hibernoma and any combinations thereof.

Without wishing to be bound by any theory or mechanism of action, it is contemplated that the CCH degrades fibrotic tissue in the subject, while the compound of formula (I) induces cell death in fat tissue of the subject. In some embodiments, addition of CCH may enhance therapeutic effect of a compound of formula (I) by degrading fibrotic tissues in the fat tissue prior to administration of the compound of formula (I). In some embodiments, CCH is administered to a fatty tissue under conditions sufficient to degrade fibrotic tissue in the fat tissue. A compound of formula (I) is subsequently injected (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48 hours after administration of CCH), thereby inducing fat cell death. In some embodiments, addition of CCH may enhance the effect of a compound of formula (I) at the injection site, as less fibrotic tissue is present.

In some embodiments, a compound of formula (I) has a cytotoxic effects on fat cells in the subject. After injection of a therapeutically-effective amount of a compound of formula (I) into fat cells, the compound may diffuse into adjacent tissue (e.g., non-fat tissue), causing undesired cytotoxicity. As demonstrated herein, addition of CCH may attenuate compound (I)-induced cytotoxicity in cells adjacent to the fat tissue. In some embodiments, a combination of compound (I) and CCH is injected into fat tissue, at concentrations sufficient to kill fat cells at the injection site. Both compounds diffuse into adjacent tissues at lower concentrations, whereby the CCH protects cells (e.g., non-fat cells) in said adjacent tissue from compound (I)-induced cell death. Thus, a combination of CCH and compound (I) on the one hand has improved fat tissue cytotoxicity, while on the other hand reduces cytotoxicity in non-injected tissue at the vicinity of the injection site, thus minimizing the potential for undesirable side effects at injection-adjacent non-fat tissues, and providing an improved side-effect profile.

In some embodiments, the concentration of CCH sufficient to attenuate compound (I)-induced cell death is about 0.1 uM to about 1 uM, e.g., 0.1 uM, 0.15 uM, 0.2 uM, 0.25 uM, 0.3 uM, 0.35 uM, 0.4 uM, 0.45 uM, 0.5 uM, 0.55, uM, 0.6 uM, 0.65 uM, 0.7 uM, 0.75 uM, 0.8 uM, 0.85 uM, 0.9 uM, 0.95 uM or 1 uM.

### Dosing and Dosing Regimens

In some embodiments, the pharmaceutical composition is formulated for subcutaneous injection directly into fat cells or fatty tissue. In some embodiments, the compound of formula (I) is administered to fat tissue in the subject at a concentration of least about 1 uM. In some embodiments, the compound of formula (I) is administered to fat tissue in the subject at a concentration from about 1 uM to about 1,000 uM. In some embodiments, the compound of formula (I) is administered to fat tissue in the subject at a concentration from about 1 uM, 10 uM, 20 uM, 30 uM, 40 uM, 50 uM, 60 uM, 70 uM, 80 uM, 90 uM, 100 uM, 110 uM, 120 uM, 130 uM, 140 uM, 150 uM, 160 uM, 170 uM, 180 uM, 190 uM, 200 uM, 210 uM, 220 uM, 230 uM, 240 uM, 250 uM, 260 uM, 270 uM, 280 uM, 290 uM, 300 uM, 310 uM, 320 uM, 330 uM, 340 uM, 350 uM, 3160 uM, 370 uM, 380 uM, 390 uM, 400 uM, 410 uM, 420 uM, 430 uM, 440 uM, 450 uM, 460 uM, 470 uM, 480 uM, 490 uM, 500 uM, 510 uM, 520 uM, 530 uM, 540 uM, 550 uM, 560 uM, 570 uM, 580 uM, 590 uM, 600 uM, 610 uM, 620 uM, 630 uM, 640 uM, 650 uM, 660 uM, 670 uM, 6180 uM, 690 uM, 700 uM, 710 uM, 720 uM, 730 uM, 740 uM, 750 uM, 760 uM, 770 uM, 780 uM, 790 uM, 800 uM, 810 uM, 820 uM, 830 uM, 840 uM, 850 uM, 860 uM, 870 uM, 880 uM, 890 uM, 900 uM, 910 uM, 920 uM, 930 uM, 940 uM, 950 uM, 960 uM, 970 uM, 980 uM, 990 uM, or 1,000 uM. In some embodiments, the compound of formula (I) is administered to fat tissue in the subject at a concentration of about 100 uM. In some embodiments, the compound of formula (I) is administered to fat tissue in the subject at a concentration of about 100 uM to about 300 uM.

In some embodiments, the compound of formula (I) is administered to fat tissue in the subject at a dosage of from about 1 to about 10 mg compound (I) (e.g., Compound 1, 2, 3, 4). In some embodiments, the pharmaceutical composition is formulated for subcutaneous injection directly into fatty tissue at a dosage of from about 1 to about 50 mg compound (I) (e.g., compound 1, 2, 3, 4), e.g., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, or 50 mg compound (I) (e.g., compound 1, 2, 3, 4).

In some embodiments, the CCH is administered to fat tissue in the subject at a concentration of greater than about 1 uM. In some embodiments, the CCH is administered to fat tissue in the subject at a concentration of about 1 uM to about 10 uM. In some embodiments, the CCH is administered to fat tissue in the subject at a concentration of about 1 uM, about 2 uM, about 3 uM, about 4 uM, about 5 uM, about 6 uM, about 7 uM, about 8 uM, about 9 uM, or about 10 uM, In some embodiments, the CCH is administered to fat tissue in the subject at a concentration of about 3 uM.

In some embodiments, each dose of the CCH is about 1 mg or less. In some embodiments, each dose of the CCH is about 0.05, 0.1, 0.15, 0.2, 0.25, 0.3, 0.35, 0.4, 0.45, 0.5, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.855, 0.86, 0.87, 0.88, 0.89, 0.90, 0.91, 0.92, 0.93, 0.94, 0.95, 0.96, 0.97, 0.98, 0.99, or 1 mg. In some embodiments, each dose of the CCH is about 0.84 mg. In some embodiments, each dose of the CCH is about 0.92 mg.

In some embodiments, each dose of the CCH is about 0.1 milligrams to about 1 milligram. In some embodiments, each dose of the CCH is about 0.1 milligrams to about 0.2 milligrams, about 0.1 milligrams to about 0.3 milligrams, about 0.1 milligrams to about 0.4 milligrams, about 0.1 milligrams to about 0.5 milligrams, about 0.1 milligrams to about 0.6 milligrams, about 0.1 milligrams to about 0.7 milligrams, about 0.1 milligrams to about 0.8 milligrams, about 0.1 milligrams to about 0.9 milligrams, about 0.1 milligrams to about 1 milligram, about 0.2 milligrams to about 0.3 milligrams, about 0.2 milligrams to about 0.4 milligrams, about 0.2 milligrams to about 0.5 milligrams, about 0.2 milligrams to about 0.6 milligrams, about 0.2 milligrams to about 0.7 milligrams, about 0.2 milligrams to about 0.8 milligrams, about 0.2 milligrams to about 0.9 milligrams, about 0.2 milligrams to about 1 milligram, about 0.3 milligrams to about 0.4 milligrams, about 0.3 milligrams to about 0.5 milligrams, about 0.3 milligrams to about 0.6 milligrams, about 0.3 milligrams to about 0.7 milligrams, about 0.3 milligrams to about 0.8 milligrams, about 0.3 milligrams to about 0.9 milligrams, about 0.3 milligrams to about 1 milligram, about 0.4 milligrams to about 0.5 milligrams, about 0.4 milligrams to about 0.6 milligrams, about 0.4 milligrams to about 0.7 milligrams, about 0.4 milligrams to about 0.8 milligrams, about 0.4 milligrams to about 0.9 milligrams, about 0.4 milligrams to about 1 milligram, about 0.5 milligrams to about 0.6 milligrams, about 0.5 milligrams to about 0.7 milligrams, about 0.5 milligrams to about 0.8 milligrams, about 0.5 milligrams to about 0.9 milligrams, about 0.5 milligrams to about 1 milligram, about 0.6 milligrams to about 0.7 milligrams, about 0.6 milligrams to about 0.8 milligrams, about 0.6 milligrams to about 0.9 milligrams, about 0.6 milligrams to about 1 milligram, about 0.7 milligrams to about 0.8 milligrams, about 0.7 milligrams to about 0.9 milligrams, about 0.7 milligrams to about 1 milligram, about 0.8 milligrams to about 0.9 milligrams, about 0.8 milligrams to about 1 milligram, or about 0.9 milligrams to about 1 milligram. In some embodiments, each dose of the CCH is about 0.1 milligrams, about 0.2 milligrams, about 0.3 milligrams, about 0.4 milligrams, about 0.5 milligrams, about 0.6 milligrams, about 0.7 milligrams, about 0.8 milligrams, about 0.9 milligrams, or about 1 milligram. In some embodiments, each dose of the CCH is at least about 0.1 milligrams, about 0.2 milligrams, about 0.3 milligrams, about 0.4 milligrams, about 0.5 milligrams, about 0.6 milligrams, about 0.7 milligrams, about 0.8 milligrams, or about 0.9 milligrams. In some embodiments, each dose of the CCH is at most about 0.2 milligrams, about 0.3 milligrams, about 0.4 milligrams, about 0.5 milligrams, about 0.6 milligrams, about 0.7 milligrams, about 0.8 milligrams, about 0.9 milligrams, or about 1 milligram.

The total amount of each compound administered will be dependent on the mammal being treated, the severity of the disorder or condition, the rate of administration, the disposition of the compound and the discretion of the prescribing physician. However, an effective dosage may be in the range of about 0.001 to about 100 mg per kg body weight per day, in single or divided doses. In some embodiments, a compound is administered in an amount ranging from about 0.01 mg/kg to about 100 mg/kg, 0.1 mg/kg to about 100 mg/kg, about 10 mg/kg to about 80 mg/kg, about 20 mg/kg to about 50 mg/kg, and the like. In some instances, dosage levels below the lower limit of the aforesaid range may be more than adequate, while in other cases still larger doses may be employed without causing any harmful side effect, e.g., by dividing such larger doses into several small doses for administration throughout the day. In some embodiments, an effective dosage may be provided in pulsed dosing (i.e., administration of the compound in consecutive days, followed by consecutive days of rest from administration).

In some embodiments, the compound of Formula (I) may be administered in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg per subject.

In some embodiments, the compound of Formula (I) may be administered in an amount of about 1 milligram to about 1,000 milligrams. In some embodiments, the compound of Formula (I) may be administered in an amount of about 1 milligram to about 2 milligrams, about 1 milligram to about 5 milligrams, about 1 milligram to about 10 milligrams, about 1 milligram to about 25 milligrams, about 1 milligram to about 50 milligrams, about 1 milligram to about 100 milligrams, about 1 milligram to about 250 milligrams, about 1 milligram to about 500 milligrams, about 1 milligram to about 750 milligrams, about 1 milligram to about 1,000 milligrams, about 2 milligrams to about 5 milligrams, about 2 milligrams to about 10 milligrams, about 2 milligrams to about 25 milligrams, about 2 milligrams to about 50 milligrams, about 2 milligrams to about 100 milligrams, about 2 milligrams to about 250 milligrams, about 2 milligrams to about 500 milligrams, about 2 milligrams to about 750 milligrams, about 2 milligrams to about 1,000 milligrams, about 5 milligrams to about 10 milligrams, about 5 milligrams to about 25 milligrams, about 5 milligrams to about 50 milligrams, about 5 milligrams to about 100 milligrams, about 5 milligrams to about 250 milligrams, about 5 milligrams to about 500 milligrams, about 5 milligrams to about 750 milligrams, about 5 milligrams to about 1,000 milligrams, about 10 milligrams to about 25 milligrams, about 10 milligrams to about 50 milligrams, about 10 milligrams to about 100 milligrams, about 10 milligrams to about 250 milligrams, about 10 milligrams to about 500 milligrams, about 10 milligrams to about 750 milligrams, about 10 milligrams to about 1,000 milligrams, about 25 milligrams to about 50 milligrams, about 25 milligrams to about 100 milligrams, about 25 milligrams to about 250 milligrams, about 25 milligrams to about 500 milligrams, about 25 milligrams to about 750 milligrams, about 25 milligrams to about 1,000 milligrams, about 40 milligrams to about 50 milligrams, about 40 milligrams to about 100 milligrams, about 40 milligrams to about 250 milligrams, about 40 milligrams to about 500 milligrams, about 40 milligrams to about 750 milligrams, about 40 milligrams to about 1,000 milligrams, about 50 milligrams to about 100 milligrams, about 50 milligrams to about 250 milligrams, about 50 milligrams to about 500 milligrams, about 50 milligrams to about 750 milligrams, about 50 milligrams to about 1,000 milligrams, about 100 milligrams to about 250 milligrams, about 100 milligrams to about 500 milligrams, about 100 milligrams to about 750 milligrams, about 100 milligrams to about 1,000 milligrams, about 250 milligrams to about 500 milligrams, about 250 milligrams to about 750 milligrams, about 250 milligrams to about 1,000 milligrams, about 500 milligrams to about 750 milligrams, about 500 milligrams to about 1,000 milligrams, or about 750 milligrams to about 1,000 milligrams. In some embodiments, the compound of Formula (I) may be administered in an amount of about 1 milligram, about 2 milligrams, about 5 milligrams, about 10 milligrams, about 25 milligrams, about 40 milligrams, about 50 milligrams, about 100 milligrams, about 250 milligrams, about 500 milligrams, about 750 milligrams, or about 1,000 milligrams. In some embodiments, the compound of Formula (I) may be administered in an amount of about at least about 1 milligram, about 2 milligrams, about 5 milligrams, about 10 milligrams, about 25 milligrams, about 50 milligrams, about 100 milligrams, about 250 milligrams, about 500 milligrams, or about 750 milligrams. In some embodiments, the compound of Formula (I) may be administered in an amount of about at most about 2 milligrams, about 5 milligrams, about 10 milligrams, about 25 milligrams, about 50 milligrams, about 100 milligrams, about 250 milligrams, about 500 milligrams, about 750 milligrams, or about 1,000 milligrams. These amounts can be single doses administered singly or split across multiple administrations.

In some embodiments, the pharmaceutical composition is administered in a single dose. In some embodiments, the pharmaceutical composition is administered via a single injection. In some embodiments, the pharmaceutical composition is administered in a single dose via a single injection. In some embodiments, the pharmaceutical composition is administered in a single dose via multiple injections. In some embodiments, the pharmaceutical composition is administered in multiple doses. the pharmaceutical composition is administered in multiple doses via multiple injections.

In some embodiments, the pharmaceutical composition is administered via multiple injections, e.g., 2, 3, 4, 5, 6, 7, 8, 9, 10, 11, 12, 13, 14, or 15 injections. In some embodiments, the pharmaceutical composition is administered via multiple injections, e.g., 2, 3, 4, 5, 6, 7, 8, 9, 10, 11, 12, 13, 14, 15, 16, 17 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 injections per subject. In some embodiments, the pharmaceutical composition is administered in a single dose via multiple injections, e.g., 2, 3, 4, 5, 6, 7, 8, 9, 10, 11, 12, 13, 14, or 15 injections. In some embodiments, the pharmaceutical composition is administered in a single dose via multiple injections, e.g., 2, 3, 4, 5, 6, 7, 8, 9, 10, 11, 12, 13, 14, 15, 16, 17 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 injections per subject.

In some embodiments, each injection contains about 0.1 milligram to about 100 milligrams of a compound of Formula (I). In some embodiments, each injection contains about 1 milligram to about 100 milligrams, about 1 milligram to about 50 milligrams, about 1 milligram to about 40 milligrams, about 1 milligram to about 30 milligrams, about 1 milligram to about 25 milligrams, about 1 milligram to about 20 milligrams, about 1 milligram to about 15 milligrams, about 1 milligram to about 10 milligrams, about 1 milligram to about 7.5 milligrams, about 1 milligram to about 5 milligrams, about 2 milligrams to about 25 milligrams, about 2 milligrams to about 20 milligrams, about 2 milligrams to about 15 milligrams, about 2 milligrams to about 10 milligrams, about 2 milligrams to about 7.5 milligrams, about 2 milligrams to about 5 milligrams, about 3 milligrams to about 25 milligrams, about 3 milligrams to about 20 milligrams, about 3 milligrams to about 15 milligrams, about 3 milligrams to about 10 milligrams, about 3 milligrams to about 7.5 milligrams, about 3 milligrams to about 5 milligrams, about 4 milligrams to about 25 milligrams, about 4 milligrams to about 20 milligrams, about 4 milligrams to about 15 milligrams, about 4 milligrams to about 10 milligrams, about 4 milligrams to about 7.5 milligrams, about 4 milligrams to about 5 milligrams, about 5 milligrams to about 25 milligrams, about 5 milligrams to about 20 milligrams, about 5 milligrams to about 15 milligrams, about 5 milligrams to about 10 milligrams, or about 5 milligrams to about 7.5 milligrams of a compound of Formula (I). In some embodiments, each injection be administered in an amount of about 1 milligram, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg per injection.

The compositions comprising CCH and Formula I can be administered separately or together. Administration can be made locally at or adjacent to the site of a fat related condition such as a lipoma, angiolipoma, a fatty tumor, a Dercum disease lesion, lipedema, hibernoma. In certain embodiments, CCH is administered at least 12, 24, 48, or 72 hours prior to administration of a compound of Formula I. A dose of CCH may be administered 1, 2, 3, 4, 5, 6, 7, 8, 9, 10 times or more before administration of a compound of Formula I. Successive administrations of CCH or a compound of Formula I may be made after intervals of at least 12 hours, 24 hours, 48 hours, 72 hours, 1 week, 2 weeks, 3 weeks, or 4 weeks.

### Pharmaceutical Compositions

In some embodiments, the compound of formula (I) is provided in a pharmaceutical composition in unit dosage form, wherein the unit dosage form further comprises at least one pharmaceutically-acceptable excipient. In some embodiments, the pharmaceutical composition is in a form of a cream, a gel, an ointment, an aqueous solution, an oil-in-water solution, a foam, a patch, a tape, a bandage, a sponge and or combinations thereof. In some embodiments, the pharmaceutical composition is formulated in a liquid dosage form. In some embodiments, the pharmaceutical composition is formulated for parenteral administration. In some embodiments, the pharmaceutical composition is formulated for subcutaneous injection. In some embodiments, the compound of formula (I) is formulated for at least a single injection. In some embodiments, the compound of formula (I) is formulated for multiple injections.

In some embodiments, the CCH is provided in a pharmaceutical composition in unit dosage form, wherein the unit dosage form further comprise sat least one pharmaceutically-acceptable excipient. In some embodiments, the pharmaceutical composition is in a form of a cream, a gel, an ointment, an aqueous solution, an oil-in-water solution, a foam, a patch, a tape, a bandage, a sponge and or combinations thereof. In some embodiments, is formulated for parenteral administration. In some embodiments, the pharmaceutical composition is formulated for subcutaneous injection. In some embodiments, the CCH is formulated for a single injection. In some embodiments, the CCH is formulated for multiple injections.

The pharmaceutical compositions of the disclosure may comprise additionally any other suitable substances such as other therapeutically useful substances, diagnostically useful substances, pharmaceutically acceptable carriers or the like. Thus, a composition of the present disclosure may be formulated in any suitable pharmaceutical formulation. A pharmaceutical composition of the present disclosure typically contains an active ingredient (e.g., a compound of Formula (I), or a pharmaceutically-acceptable salt and/or coordination complex thereof, and/or a Collagenase Clostridium histolyticum (CCH)), and one or more pharmaceutically-acceptable excipients or carriers, including but not limited to: inert solid diluents and fillers, diluents, sterile aqueous solution and various organic solvents, permeation enhancers, solubilizers, and adjuvants. A composition of the present disclosure may be formulated in any suitable pharmaceutical formulation.

Pharmaceutical compositions may be provided in any suitable form, which may depend on the route of administration. In some embodiments, the pharmaceutical composition disclosed herein can be formulated in dosage form for administration to a subject. In some embodiments, the pharmaceutical composition is formulated for parenteral, topical, transdermal, buccal, sublingual, subcutaneous, intramuscular, intravenous, intratumoral, and/or intraperitoneal administration. In some embodiments, the pharmaceutical composition can be formulated as a unit dosage.

In some embodiments, the composition is provided in one or more unit doses. For example, the composition can be administered in 1, 2, 3, 4, 5, 6, 7, 14, 30, 60, or more doses. Such amount can be administered each day, for example in individual doses administered once, twice, or three or more times a day. However, dosages stated herein on a per day basis should not be construed to require administration of the daily dose each and every day. For example, if one of the agents is provided in a suitably slow-release form, two or more daily dosage amounts can be administered at a lower frequency, e.g., as a depot injection administered every second day to once a month or even longer. Most typically and conveniently for the subject, a pharmaceutical composition comprising a compound of Formula (I) can be administered once a day, for example in the morning, in the evening or during the day.

Pharmaceutical compositions described herein may contain one or more pharmaceutically-acceptable excipients. The phrase “pharmaceutically-acceptable excipient” or “pharmaceutically-acceptable carrier” as used herein means a pharmaceutically-acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically-acceptable carriers include: (1) surfactants such as hydrophilic surfactants, lipophilic surfactants, non-ionic surfactants, cationic surfactants, anionic surfactants; (2) solubilizers such as alcohols; (3) solvents such as water, alcohol and glycols; (4) sugars, such as lactose, glucose and sucrose; (5) starches, such as corn starch and potato starch; (6) cellulose, and its derivatives, such as sodium carboxymethyl cellulose, ethyl cellulose and cellulose acetate; (7) powdered tragacanth; (8) malt; (9) gelatin; (10) talc; (11) excipients, such as cocoa butter and suppository waxes; (12) oils, such as peanut oil, cottonseed oil, safflower oil, sesame oil, olive oil, corn oil and soybean oil; (13) glycols, such as propylene glycol; (14) polyols, such as glycerin, sorbitol, mannitol and polyethylene glycol; (15) esters, such as ethyl oleate and ethyl laurate; (16) agar; (17) buffering agents, such as magnesium hydroxide and aluminum hydroxide; (18) alginic acid; (19) pyrogen-free water; (20) isotonic saline; (21) Ringer's solution; (22) ethyl alcohol; (23) phosphate buffer solutions; and (24) other non-toxic compatible substances employed in pharmaceutical formulations. The composition can further include one or more pharmaceutically-acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In some embodiments, the pharmaceutical composition comprises one or more surfactants. Surfactants which can be used to form pharmaceutical composition and dosage forms of the disclosure include, but are not limited to, hydrophilic surfactants, lipophilic surfactants, non-ionic surfactants, cationic surfactants, anionic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof, lysophospholipids and derivatives thereof, carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof, carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group of glycerides, vegetable oils, hydrogenated vegetable oils, fatty acids, and sterols; polyoxyethylene sterols, derivatives, and analogues thereof, polyoxyethylated vitamins and derivatives thereof, polyoxyethylene-polyoxypropylene block copolymers; and mixtures thereof, polyethylene glycol sorbitan fatty acid esters and hydrophilic transesterification products of a polyol with at least one member of the group of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, Tween 80, sucrose monostearate, sucrose monolaurate, sucrose monopalmitate, PEG 10-100 nonyl phenol series, PEG 15-100 octyl phenol series, and poloxamers. In some embodiments, the surfactant is Tween. In some embodiments, the surfactant is Tween 80.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group of vegetable oils, hydrogenated vegetable oils, and triglycerides.

In one embodiment, the composition may include a solvent/solubilizer to ensure good solubilization and/or dissolution of the compound of the present disclosure and to minimize precipitation of the compound of the present disclosure. This can be especially important for injection. A solubilizer may also be added to increase the solubility of the hydrophilic drug and/or other components, such as surfactants, or to maintain the composition as a stable or homogeneous solution or dispersion. Examples of suitable solvents/solubilizers include, but are not limited to: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof, and other solubilizers known in the art, such as dimethyl acetamide, dimethyl isosorbide, N-methyl pyrrolidones, monooctanoin, diethylene glycol monoethyl ether, and water. In some embodiments, the solubilizer is benzyl alcohol.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. If present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

In addition, an acid or a base may be incorporated into the composition to facilitate processing, to enhance stability, or for other reasons. Examples of pharmaceutically-acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically-acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically-acceptable cation, such as ammonium, alkali metals, alkaline earth metals, and the like. Example may include, but not limited to, sodium, potassium, lithium, magnesium, calcium and ammonium.

Suitable acids for use in the compositions of the disclosure are pharmaceutically-acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

Provided herein, in one aspect, is a pharmaceutical composition comprising a compound of Formula (I), in combination with a Collagenase Clostridium histolyticum (CCH):

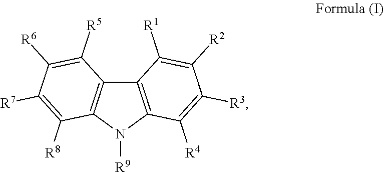

or a pharmaceutically-acceptable salt thereof, wherein:
each of R¹, R², R³, R⁴, R⁵, R⁶, R⁷, and R⁸ is independently H, halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), —ON(═O), C₁₋₅alkyl, C₂₋₅alkenyl, or C₂₋₅alkynyl; wherein each alkyl, alkenyl, or alkynyl is independently optionally substituted with one or more substituents selected from the group consisting of halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), and —ON(═O);
R⁹ is C₁₋₉alkyl, C₂₋₉alkenyl, C₂₋₉alkynyl, or a 3- to 10-membered heterocycloalkyl; wherein R⁹ is substituted with at least one quaternary ammonium group or a phosphonium group;
each R¹⁰ is independently H, C₁₋₅alkyl, C₂₋₅alkenyl, C₂₋₅alkynyl, C₁₋₅heteroalkyl, C₁₋₅haloalkyl, or C₃₋₆cycloalkyl;
each R¹¹ and R¹² is independently H, C₁₋₅alkyl, C₂₋₅alkenyl, C₂₋₅alkynyl, C₁₋₅heteroalkyl, C₁₋₅haloalkyl, or C₃₋₆cycloalkyl; or R¹¹ and R¹² together with the nitrogen atom to which they are attached is a 3- to 10-membered heterocycloalkyl which is optionally substituted; and
each R¹³ is independently H, C₁₋₅alkyl, C₂₋₅alkenyl, C₂₋₅alkynyl, C₁₋₅heteroalkyl, C₁₋₅ haloalkyl, or C₃₋₆cycloalkyl.

In some embodiments, the pharmaceutical composition comprises less than about 50% water by weight. In some embodiments, the pharmaceutical composition comprises less than about 30% water by weight. In some embodiments, the pharmaceutical composition comprises less than about 10% water by weight. In some embodiments, the pharmaceutical composition comprises from about 0% to about 30% water by weight. In some embodiments, the pharmaceutical composition comprises from about 10% to about 30% water by weight. In some embodiments, the pharmaceutical composition comprises from about 15% to about 30% water by weight. In some embodiments, the pharmaceutical composition comprises from about 15% to about 25% water by weight. In some embodiments, the pharmaceutical composition comprises from about 20% to about 30% water by weight. In some embodiments, the pharmaceutical composition comprises from about 23% to about 27% water by weight. In some embodiments, the pharmaceutical composition comprises from about 24% to about 26% water by weight. In some embodiments, the pharmaceutical composition comprises about 0% water by weight. In some embodiments, the pharmaceutical composition comprises about 1% water by weight. In some embodiments, the pharmaceutical composition comprises about 2% water by weight. In some embodiments, the pharmaceutical composition comprises about 3% water by weight. In some embodiments, the pharmaceutical composition comprises about 4% water by weight. In some embodiments, the pharmaceutical composition comprises about 5% water by weight. In some embodiments, the pharmaceutical composition comprises about 6% water by weight. In some embodiments, the pharmaceutical composition comprises about 7% water by weight. In some embodiments, the pharmaceutical composition comprises about 8% water by weight. In some embodiments, the pharmaceutical composition comprises about 9% water by weight. In some embodiments, the pharmaceutical composition comprises about 10% water by weight. In some embodiments, the pharmaceutical composition comprises about 11% water by weight. In some embodiments, the pharmaceutical composition comprises about 12% water by weight. In some embodiments, the pharmaceutical composition comprises about 13% water by weight. In some embodiments, the pharmaceutical composition comprises about 14% water by weight. In some embodiments, the pharmaceutical composition comprises about 15% water by weight. In some embodiments, the pharmaceutical composition comprises about 16% water by weight. In some embodiments, the pharmaceutical composition comprises about 17% water by weight. In some embodiments, the pharmaceutical composition comprises about 18% water by weight. In some embodiments, the pharmaceutical composition comprises about 19% water by weight. In some embodiments, the pharmaceutical composition comprises about 20% water by weight. In some embodiments, the pharmaceutical composition comprises about 21% water by weight. In some embodiments, the pharmaceutical composition comprises about 22% water by weight. In some embodiments, the pharmaceutical composition comprises about 23% water by weight. In some embodiments, the pharmaceutical composition comprises about 24% water by weight. In some embodiments, the pharmaceutical composition comprises about 25% water by weight. In some embodiments, the pharmaceutical composition comprises about 26% water by weight. In some embodiments, the pharmaceutical composition comprises about 27% water by weight. In some embodiments, the pharmaceutical composition comprises about 28% water by weight. In some embodiments, the pharmaceutical composition comprises about 29% water by weight. In some embodiments, the pharmaceutical composition comprises about 30% water by weight.

In some embodiments, the pharmaceutical composition comprises at least about 0.1% by weight of the compound of Formula (I). In some embodiments, the pharmaceutical composition comprises between about 0.1% to about 10% by weight of the compound of Formula (I). In some embodiments, the pharmaceutical composition comprises between about 1% to about 5% by weight of the compound of Formula (I). In some embodiments, the pharmaceutical composition comprises about 0.1% by weight of the compound of Formula (I). In some embodiments, the pharmaceutical composition comprises about 0.2% by weight of the compound of Formula (I). In some embodiments, the pharmaceutical composition comprises about 0.3% by weight of the compound of Formula (I). In some embodiments, the pharmaceutical composition comprises about 0.4% by weight of the compound of Formula (I). In some embodiments, the pharmaceutical composition comprises about 0.5% by weight of the compound of Formula (I). In some embodiments, the pharmaceutical composition comprises about 0.6% by weight of the compound of Formula (I). In some embodiments, the pharmaceutical composition comprises about 0.7% by weight of the compound of Formula (I). In some embodiments, the pharmaceutical composition comprises about 0.8% by weight of the compound of Formula (I). In some embodiments, the pharmaceutical composition comprises about 0.9% by weight of the compound of Formula (I). In some embodiments, the pharmaceutical composition comprises about 1% by weight of the compound of Formula (I). In some embodiments, the pharmaceutical composition comprises about 1.5% by weight of the compound of Formula (I). In some embodiments, the pharmaceutical composition comprises about 2% by weight of the compound of Formula (I). In some embodiments, the pharmaceutical composition comprises about 2.5% by weight of the compound of Formula (I). In some embodiments, the pharmaceutical composition comprises about 3% by weight of the compound of Formula (I). In some embodiments, the pharmaceutical composition comprises about 3.5% by weight of the compound of Formula (I). In some embodiments, the pharmaceutical composition comprises about 4% by weight of the compound of Formula (I). In some embodiments, the pharmaceutical composition comprises about 4.5% by weight of the compound of Formula (I). In some embodiments, the pharmaceutical composition comprises about 5% by weight of the compound of Formula (I). In some embodiments, the pharmaceutical composition comprises about 5.5% by weight of the compound of Formula (I). In some embodiments, the pharmaceutical composition comprises about 6% by weight of the compound of Formula (I). In some embodiments, the pharmaceutical composition comprises about 6.5% by weight of the compound of Formula (I). In some embodiments, the pharmaceutical composition comprises about 7% by weight of the compound of Formula (I). In some embodiments, the pharmaceutical composition comprises about 7.5% by weight of the compound of Formula (I). In some embodiments, the pharmaceutical composition comprises about 8% by weight of the compound of Formula (I). In some embodiments, the pharmaceutical composition comprises about 8.5% by weight of the compound of Formula (I). In some embodiments, the pharmaceutical composition comprises about 9% by weight of the compound of Formula (I). In some embodiments, the pharmaceutical composition comprises about 9.5% by weight of the compound of Formula (I). In some embodiments, the pharmaceutical composition comprises about 10% by weight of the compound of Formula (I).

In some embodiments, the compound of Formula (I) can be present in a composition in an amount of about 10 mg/mL. In some embodiments, the compound of Formula (I) can be present in a composition in an amount of about 20 mg/mL. In some embodiments, the compound of Formula (I) can be present in a composition in an amount of about 25 mg/mL. In some embodiments, the compound of Formula (I) can be present in a composition in an amount of about 50 mg/mL. In some embodiments, the compound of Formula (I) can be present in a composition in an amount of about 75 mg/mL. In some embodiments, the compound of Formula (I) can be present in a composition in an amount of about 100 mg/mL. In some embodiments, the compound of Formula (I) can be present in a composition in an amount of about 150 mg/mL. In some embodiments, the compound of Formula (I) can be present in a composition in an amount of about 200 mg/mL. In some embodiments the compound of Formula (I) can be present in a composition in an amount of about 250 mg/mL. In some embodiments, the compound of Formula (I) can be present in a composition in an amount of about 400 mg/mL.

In some embodiments, the compound of Formula (I) can be present in a composition in an amount of about 1 mg, about 2 mg, about 3 mg, about 4 mg, about 5 mg, about 7.5 mg, about 10 mg, about 12.5 mg, about 15 mg, about 17.5 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

In some embodiments, CCH can be present in a composition in an amount of about 10 mg/mL. In some embodiments, CCH can be present in a composition in an amount of about 0.1 to about 10 mg/ml. In some embodiments, CCH can be present in a composition in an amount of about 0.2 to about 10 mg/ml. In some embodiments, CCH can be present in a composition in an amount of about 0.1 to about 5 mg/ml. In some embodiments, CCH can be present in a composition in an amount of about 0.1 to about 1 mg/ml. In some embodiments, CCH can be present in a composition in an amount of about 0.1 to about 0.5 mg/ml. In some embodiments, CCH can be present in a composition in an amount of about 0.2 to about 1 mg/ml. In some embodiments, CCH can be present in a composition in an amount of about 0.23 to about 1 mg/ml. In some embodiments, CCH can be present in a composition in an amount of about 0.25 to about 1 mg/ml. In some embodiments, CCH can be present in a composition in an amount of about 0.3 to about 1 mg/ml. In some embodiments, CCH can be present in a composition in an amount of about 0.4 to about 1 mg/ml. In some embodiments, CCH can be present in a composition in an amount of about 0.2 mg/mL In some embodiments, CCH can be present in a composition in an amount of about 0.23 mg/mL. In some embodiments, CCH can be present in a composition in an amount of about 0.25 mg/mL. In some embodiments, CCH can be present in a composition in an amount of about 0.3 mg/mL. In some embodiments, CCH can be present in a composition in an amount of about 0.4 mg/mL In some embodiments, CCH can be present in a composition in an amount of about 0.5 mg/mL. In some embodiments, CCH can be present in a composition in an amount of about 1 mg/mL. In some embodiments, CCH can be present in a composition in an amount of about 1.5 mg/mL. In some embodiments, CCH can be present in a composition in an amount of about 2.0 mg/mL. In some embodiments, CCH can be present in a composition in an amount of about 2.1 mg/mL. In some embodiments, CCH can be present in a composition in an amount of about 2.2 mg/mL. In some embodiments CCH can be present in a composition in an amount of about 2.3 mg/mL. In some embodiments, CCH can be present in a composition in an amount of about 2.4 mg/mL. In some embodiments, CCH can be present in a composition in an amount of about 2.5 mg/mL. In some embodiments, CCH can be present in a composition in an amount of about 2.6 mg/mL. In some embodiments, CCH can be present in a composition in an amount of about 2.7 mg/mL. In some embodiments, CCH can be present in a composition in an amount of about 2.8 mg/mL. In some embodiments, CCH can be present in a composition in an amount of about 2.9 mg/mL. In some embodiments, CCH can be present in a composition in an amount of about 3.0 mg/mL. In some embodiments, CCH can be present in a composition in an amount of about 3.5 mg/mL. In some embodiments, CCH can be present in a composition in an amount of about 4.0 mg/mL. In some embodiments, CCH can be present in a composition in an amount of about 4.5 mg/mL. In some embodiments, CCH can be present in a composition in an amount of about 5.0 mg/mL.

### Pharmaceutical Compositions for Injection

In some embodiments, the disclosure provides a pharmaceutical composition for injection containing a compound of Formula (I) and a pharmaceutical excipient suitable for injection. Components and amounts of agents in the composition are as described herein.

In some embodiments, the disclosure provides a pharmaceutical composition for injection containing a Collagenase Clostridium histolyticum (CCH) and a pharmaceutical excipient suitable for injection. Components and amounts of agents in the composition are as described herein.

The forms in which a composition of the present disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. In some embodiments, the compositions comprise solvents (e.g., water, alcohols, glycols), solubilizing agents, solvents, and surfactants.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present disclosure in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

In some embodiments, the pharmaceutical composition is formulated for parenteral, topical, transdermal, buccal, sublingual, subcutaneous, intramuscular, intravenous, intratumoral, and/or intraperitoneal administration. In some embodiments, the pharmaceutical composition is formulated for parenteral administration. In some embodiments, the pharmaceutical composition is formulated for injection. In some embodiments, the pharmaceutical composition is formulated for intratumoral injection. In some embodiments, the pharmaceutical composition is formulated as an injection, a patch, a cream, a gel, or an ointment.

### Kits

The disclosure also provides kits. The kits may include a pharmaceutical composition comprising a compound of Formula (I) and/or a Collagenase Clostridium histolyticum (CCH) and one or more additional agents in suitable packaging with written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another agent. In some embodiments, the compound of the present disclosure and the agent are provided as separate compositions in separate containers within the kit. In some embodiments, the compound of the present disclosure and the agent are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

### EXAMPLES

### Example 1: Combined Cytotoxic Effect of Compound of Formula (I) and Collagenase

The objective of this in vitro study was to evaluate the combined cytotoxic effects of RZL-12 (5-(3,6-dibromo-9H-carbazol-9-yl)-N,N,N-trimethylpentan-1-aminium, provided as a chloride salt, also designated Compound 1A), and Collagenase on Wi-38 fibroblasts and on human visceral preadipocytes using Cell Titer Glo 2.0 cytotoxicity assay.

### Assay Principle

Human visceral preadipocytes were seeded in 96-well white clear bottom plates at 10,000 cells per well in 100 μl pre-adipocyte medium and incubated overnight at 37° C. 5% CO2. On the following day, adipogenic differentiation was induced by addition of 100 μl/well differentiation medium and incubation continued for 5 days. Adipogenic differentiation was verified by microscopic evaluation, addition of adipored reagents and fluorescent measurement at Ex485 nm/Em572 nm. For the treatments, test compound RZL-012 (compound 1) was prepared in DMSO at 500×stock solutions. Collagenase was prepared in HBSS at 500×stock solutions. Immediately before addition, cells' medium was replaced with 90 μl differentiation medium and test compound RZL-012 and/or collagenase were added in triplicates (each 5 μl/well). Collagenase was added about 5 minutes before RZL-012. Staurosporine (1 μM) and 0.2% DMSO were used as controls. Plates were incubated at 37° C. 5% CO₂ for 2, 6 and 24 hours. Following incubation, cytotoxicity was measured using Cell Titer Glo 2.0 (Promega).

### Materials

Test Items:

| | | | | Expiration | Storage |
|---|---|---|---|---|---|
| Name | Supplier | Catalog # | Batch No | Date | Temp. |
| RZL-012 | Raziel | NA | NA | NA | RT |
| Collagenase | SIGMA | C1639 | 049M4140V | Mar. 23 | −20° C. |

### Preparation of Test Items:

Test Item RZL-12 (1-300 μM) was prepared on the day of the testing as 150 mM stock solutions in DMSO. The 150 mM represents a 500×stock of the highest desired tested concentration. The 150 mM stock solution was first serially diluted in DMSO to acquire 500×stock solutions for each of the RZL-012 concentrations. The 500×stock solutions were then diluted 1:25 in medium to acquire 20×intermediate stock solutions. Finally, the 20×intermediate stock solutions were further diluted 1:20 in the assay (on the plate) that included seeded cells.

Test Item Collagenase (0.1-30 μM) was prepared on the day of the testing as 600 μM stock solutions in Hank's Balanced Salt Solution (HBSS). The 600 μM represents a 20× stock of the highest desired tested concentration. The 600 μM stock solution was first serially diluted in HBSS to acquire 20×intermediate stock solutions for each of the Collagenase concentrations. Finally, the 20×intermediate stock solutions were further diluted 1:20 in the assay plate that included seeded cells.

### Study Design —Cell Culture:

Human visceral pre-adipocytes (HPrAD-vis): HPrAD-vis cells (Lonza cat #PT-5005) were thawed at 37° C. and transferred into 50 ml tubes containing pre-adipocytes PGM-2 growth medium (PBM-2, 10% FBS, 1-glutamine, GA-1000). Cells were centrifuged at 300 g for 10 minutes, the supernatant was removed (Leaving about 1 ml of wash medium for not disrupting the cells) and 2 ml fresh PGM-2 medium was added. After resuspension, cells were counted and seeded in T-75 and T25 flasks at a density of 9,000 cells/cm² (665,000 cells in T75 flask, 225,000 cells/T25 flask). Medium was changed every 2-3 days until reached confluence of 70%.

Cells were sub-cultured only once, as soon as they reached 70% confluence. For this purpose, the culture medium of each flask was removed and cells were washed once with 5 ml PBS. The flask was shaken to ensure that the entire surface was covered and the PBS was removed. Then, 3 ml/T75 flask or 1 ml/T25 flask of fresh dedicated Trypsin/EDTA solution (from Lonza) was added and the flasks were incubated for 3-4 minutes at RT until ˜90% cells were detached. The Trypsin/EDTA solution was then neutralized by addition of 6 ml/T75 flask or 3 ml/T25 flask of full PGM-2 growth medium. Cells were collected in 50 ml tubes and centrifuged 10 minutes at 300 g. The supernatant was removed and the cells were resuspended in 3 ml fresh medium. Cells were then counted using a haemacytometer slide after being diluted 1:2 in trypan blue (SIGMA). For sub-culturing, cells were seeded in new T75 flasks at a density of 9,000 cells/cm² containing 15 ml fresh growth medium. Alternatively, the cells were seeded in 96-well culture plates at a density of 10,000 cells/well for the cell treatments.

### Cells Treatments:

HPrAD-vis cells: HPrAD-vis passaged once were seeded in 96-well plates as described above (10000cells/well in 100 μl medium) and allowed to settle overnight at 37° C., 5% CO2. Following day, adipogenic differentiation was induced by addition of 100 μl/well differentiation medium and incubated for 5 days. Adipogenic differentiation was verified by microscopic evaluation, addition of adipored reagents and fluorescent measurement at Ex485 nm/Em572 nm. In the morning of day 6, test and reference items were prepared as described above. Cells' medium was replaced with 90 μl fresh differentiation medium. Collagenase was added first and 5 minutes later RZL-012 was added on the cells. Staurosporine (1 μM) and 0.2% DMSO were used as controls. Plates were incubated at 37° C. 5% CO2 for 2, 6 and 24 hours. Following incubation, cell viability was determined using the CellTiter-Gg® viability reagent (Promega) with reading of plates for luminescence. Wells containing only medium were used as blank control for the CellTiter Glo assay.

Data analysis: Average data from “only medium” blank wells was subtracted from all wells. Data was normalized to the untreated cells control and represented as percent of cell death. Then, the average and standard deviation was calculated for each of the treatments. Wherever possible data was further analyzed with 4-parameters logistic curve statistical analysis using GraphPad Prism 6.0.

Results: As shown in Table 1 below, the cytotoxic IC50 for RZL-012 was determined at about 30 μM while that of collagenase was set at 0.3 μM. When combined the IC50 was 0.02 μM indicating a significant synergistic cytotoxic effect when both compounds are combined.

TABLE 1
IC50 values of RZL-12 in the presence of Collagenase after 2-, 6- and 24-hours incubation.

| | | Collagenase | Collagenase | Collagenase | Collagenase | Collagenase | Collagenase | Collagenase |
|---|---|---|---|---|---|---|---|---|
| | | 30 μM | 10 μM | 3 μM | 1 μM | 0.3 μM | 0.1 μM | 0 |
| 2 h | IC50 | ~93.20 | 71.33 | ~31.21 | 32.56 | 35.72 | 32.37 | 31.42 |
| | (μM) | | | | | | | |
| 6 h | IC50 | 38.8 | 36.45 | ~30.46 | 44.17 | 45.06 | 44.3 | 34.62 |
| | (μM) | | | | | | | |
| 24 h | IC50 | 28.84 | 28.8 | 22.87 | ~36.14 | 38.46 | 37.97 | 22.78 |
| | (μM) | | | | | | | |

The bolded numbers represent collagenase-induced increase in RZL-012 IC₅₀ vs no collagenase

TABLE 2
Average RZL-012 and collagenase induced cell death (% vs untreated cells) at 2 h of incubation

| RZL12 | Collagenase | Collagenase | Collagenase | Collagenase | Collagenase | Collagenase | Collagenase |
|---|---|---|---|---|---|---|---|
| [μM] | 30 μM | 10 μM | 3 μM | 1 μM | 0.3 μM | 0.1 μM | 0 |
| 300 | 89.5 | 89.2 | 96.6 | 99.1 | 99.6 | 99.6 | 99.9 |
| 100 | 76.4 | 72.4 | 97.4 | 98.9 | 99.3 | 99.5 | 99.7 |
| 30 | 46.0 | 33.2 | 49.7 | 45.8 | 35.0 | 43.8 | 50.4 |
| 10 | 45.0 | 22.3 | 17.7 | 7.8 | 5.0 | 4.9 | 10.4 |
| 3 | 47.6 | 26.4 | 18.0 | 6.1 | 1.1 | −1.6 | 5.9 |
| 1 | 48.0 | 29.9 | 19.2 | 2.0 | 3.0 | 6.5 | 7.0 |
| 0.01 | 46.4 | 30.3 | 20.5 | 5.3 | 0.8 | 6.5 | 10.0 |

TABLE 3
Average RZL-012 and collagenase induced cell death (% vs untreated cells) at 24 h of incubation

| RZL12 | Collagenase | Collagenase | Collagenase | Collagenase | Collagenase | Collagenase | Collagenase |
|---|---|---|---|---|---|---|---|
| [μM] | 30 μM | 10 μM | 3 μM | 1 μM | 0.3 μM | 0.1 μM | 0 |
| 300 | 100.01 | 99.82 | 99.76 | 99.96 | 99.74 | 99.67 | 100.00 |
| 100 | 99.99 | 99.92 | 99.89 | 99.65 | 99.60 | 99.66 | 99.89 |
| 30 | 90.00 | 83.68 | 79.97 | 31.37 | 23.76 | 28.35 | 72.72 |
| 10 | 80.04 | 65.12 | 33.99 | 16.08 | 5.50 | 9.43 | 12.80 |
| 3 | 77.35 | 63.40 | 27.36 | 16.21 | 2.09 | 3.62 | 6.40 |
| 1 | 78.45 | 64.29 | 28.74 | 16.48 | −0.25 | 8.18 | 4.88 |
| 0 | 79.93 | 65.68 | 33.27 | 25.57 | 6.53 | 7.28 | 7.97 |

In Tables 2 and 3, the bolded numbers represent reductions in % cell death vs either RZL-012 or collagenase only. The bolded and underlined numbers are the suggested initial tissue concentrations of RZL-012 and collagenase allowing for maximal cell killing at the injection site and best tissue preservation in surrounding areas

### CONCLUSIONS

In both cell systems, staurosporine used as a cytotoxic reference control resulted in maximal cell death of 89% after 24 hours of incubation.

In both cell systems, control of 0.2% DMSO did not influence cell survival.

The cytotoxic effect of RZL-12 and collagenase was observed at all time points for concentrations RZL-12>30 μM and Collagenase >3 μM.

Addition of collagenase at concentrations of 0.1-1 μM together with RZL-012 to adipocytes, seemed to attenuate cell cytotoxicity vs each compound alone, at the 6 h and 24 h time points (increase in IC₅₀ values). At the 2 h time point collagenase concentrations of 10 μM and 30 μM attenuated RZL-012 cytotoxicity.

It is thus suggested to inject RZL-012 and collagenase into fat at tissue concentrations of 100-300 μM and 3 μM respectively. These compound levels will assure maximal cell killing at the injection site. Both compounds will diffuse into adjacent tissues at lower tissue concentrations. The inhibitory effect of 0.1-1 μM collagenase on RZL-012 induced cell death assures that adjacent tissues will be protected from cell death and fat cell killing will be confined to the injected area.

While some embodiments have been shown and described, various modifications and substitutions may be made thereto without departing from the spirit and scope of the invention. For example, for claim construction purposes, it is not intended that the claims set forth hereinafter be construed in any way narrower than the literal language thereof, and it is thus not intended that exemplary embodiments from the specification be read into the claims. Accordingly, it is to be understood that the present disclosure has been described by way of illustration and not limitations on the scope of the claims.

1. A combination comprising Collagenase Clostridium histolyticum (CCH) and at least one compound having the general formula (I) 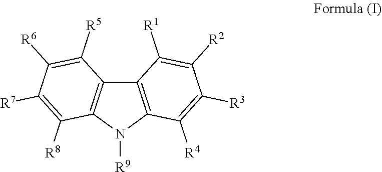
or a pharmaceutically-acceptable salt thereof, wherein:
each of R¹, R², R³, R⁴, R⁵, R⁶, R⁷, and R⁸ is independently H, halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), —ON(═O), C₁₋₅alkyl, C₂₋₅alkenyl, or C₂₋₅alkynyl; wherein each alkyl, alkenyl, or alkynyl is independently optionally substituted with one or more substituents selected from the group consisting of halogen, —CN, —NO₂, —OR¹⁰, —SR¹⁰, —S(═O)R¹⁰, —S(═O)₂R¹⁰, —NR¹¹R¹², —C(═O)NR¹¹R¹², —S(═O)NR¹¹R¹², —S(═O)₂NR¹¹R¹², —C(═O)R¹⁰, —C(═O)OR¹⁰, —NR¹³C(═O)R¹⁰, —NR¹³C(═O)NR¹¹R¹², —NR¹³S(═O)₂R¹⁰, —NR¹³S(═O)₂NR¹¹R¹², —C(═S)R¹⁰, —N(═O), —SN(═O), —NR¹³N(═O), and —ON(═O);
R⁹ is C₁₋₉alkyl, C₂₋₉alkenyl, C₂₋₉alkynyl, or a 3- to 10-membered heterocycloalkyl; wherein R⁹ is substituted with at least one quaternary ammonium group or a phosphonium group;
each R¹⁰ is independently H, C₁₋₅alkyl, C₂₋₅alkenyl, C₂₋₅alkynyl, C₁₋₅heteroalkyl, C₁₋₅haloalkyl, or C₃₋₆cycloalkyl;
each R¹¹ and R¹² is independently H, C₁₋₅alkyl, C₂₋₅alkenyl, C₂₋₅alkynyl, C₁₋₅heteroalkyl, C₁₋₅haloalkyl, or C₃₋₆cycloalkyl; or R¹¹ and R¹² together with the nitrogen atom to which they are attached is a 3- to 10-membered heterocycloalkyl which is optionally substituted; and
each R¹³ is independently H, C₁₋₅alkyl, C₂₋₅alkenyl, C₂₋₅alkynyl, C₁₋₅heteroalkyl, C₁₋₅haloalkyl, or C₃₋₆cycloalkyl.

2. The combination according to claim 1, wherein R⁹ is a straight or branched C₁-C₉ alkyl substituted with at least one quaternary amino group.

3. The combination according to claim 1, wherein said at least one quaternary amino group has a general formula (V): 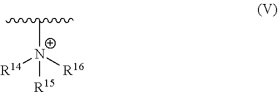
wherein each of R¹⁴, R¹⁵, and R¹⁶ is independently straight or branched C₁-C₉ alkyl, straight or branched C₂-C₉alkenyl or straight or branched C₂-C₉alkynyl.

4. (canceled)

5. (canceled)

6. The combination according to claim 1, wherein each of R¹⁴, R¹⁵, and R¹⁶ is independently a straight or branched C₁-C₉ alkyl.

7. (canceled)

8. (canceled)

9. (canceled)

10. The combination according to claim 1, wherein at least one of R¹, R², R³, and R⁴ is a halogen and at least one of R⁵, R⁶, R⁷, and R⁸ is a halogen.

11. The combination according to claim 10, wherein said halogen is bromo.

12. (canceled)

13. (canceled)

14. (canceled)

15. The combination according to claim 1, wherein said at least one compound of formula (I) is:
3-(3,6-dibromo-9H-carbazol-9-yl)-N,N,N-trimethylpropan-1-aminium;
5-(9H-carbazol-9-yl)-N,N,N-trimethylpentan-1-aminium;
5-(2-hydroxy-9H-carbazol-9-yl)-N,N,N-trimethylpentan-1-aminium; or
5-(3,6-dibromo-9H-carbazol-9-yl)-N,N,N-trimethylpentan-1-aminium,
or any salt thereof.

16. (canceled)

17. The combination according to claim 1, wherein the compound of Formula (I) is 5-(3,6-dibromo-9H-carbazol-9-yl)-N,N,N-trimethylpentan-1-aminium chloride.

18. (canceled)

19. The combination according to claim 1, wherein the combination comprises from about 0.1% to about 10% by weight of said at least one compound of formula (I).

20. (canceled)

21. (canceled)

22. (canceled)

23. (canceled)

24. (canceled)

25. The combination according to any one of the preceding claims wherein the combination comprises at least about 0.1% by weight of said Collagenase Clostridium histolyticum (CCH).

26. The combination according to claim 1, wherein CCH and said at least one compound of formula (I) are administered separately.

27. The combination according to claim 1, wherein CCH and said at least one compound of formula (I) are administered sequentially, in any order.

28. (canceled)

29. (canceled)

30. (canceled)

31. (canceled)

32. (canceled)

33. (canceled)

34. (canceled)

35. The combination according to claim 1, wherein said CCH and at least one compound of general formula (I) are formulated in a single composition.

36. The combination according to claim 1, wherein said CCH and at least one compound of general formula (I) are formulated in separate compositions.

37. The combination according to claim 1, wherein said compound of formula (I) is provided in a pharmaceutical composition in unit dosage form, wherein the unit dosage form further comprises at least one pharmaceutically-acceptable excipient.

38. (canceled)

39. (canceled)

40. The combination according to claim 37, wherein the pharmaceutical composition is formulated for parenteral administration.

41. (canceled)

41. (canceled)

42. (canceled)

43. (canceled)

44. (canceled

45. (canceled)

46. (canceled)

47. (canceled)

48. (canceled)

49. (canceled)

50. (canceled)

51. (canceled)

52. A method of treating a fat related condition or disorder in a patient in need thereof, said method comprising administering to said patient the combination of claim 1.

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. The method according to claim 52, wherein the compound of formula (I) induces cell death in fat tissue of the subject.

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. The method according to claim 52, wherein said fat related condition or disorder is selected from the group consisting of cellulite, excess fat in various body areas, lipomas, fatty tumor diseases, disorders associated with fat accumulation, dercum disease, lipedema, hibernoma and any combinations thereof.

102. A composition comprising the combination of claim 1 and at least one pharmaceutically-acceptable excipient.

103. (canceled)

104. (canceled)
